(19) United States
(12) Patent Application Publication
(10) Pub. No.: US 2024/0043809 A1
(43) Pub. Date: Feb. 8, 2024

(54) Intestinal organoid co-culture systems and methods for treating or preventing a disease or disorder associated with immune response-mediated tissue injury

(71) Applicant: NEW YORK UNIVERSITY, New York, NY (US)

(72) Inventor: Ken H. Cadwell, Forest Hills, NY (US)

(21) Appl. No.: 18/484,008

(22) Filed: Oct. 10, 2023

Related U.S. Application Data
(63) Continuation of application No. 17/096,322, filed on Nov. 12, 2020, now abandoned.
(60) Provisional application No. 62/935,035, filed on Nov. 13, 2019.

(51) Int. Cl. C12N 5/071; A61K 35/37

(52) U.S. Cl. CPC C12N 5/0679; A61K 35/37; C12N 2501/15; C12N 2501/11; A61K 45/06

(57) ABSTRACT

The invention provides methods for administering necroptosis or interferon signaling inhibitors for treating or preventing immune-related tissue injury in a subject having an inactivating mutation in ATG16L1. The invention also provides organoid cultures and co-cultures and methods of use for identifying therapeutic agents.

### CROSS REFERENCE TO RELATED APPLICATIONS

This application claims priority to U.S. Provisional Application No. 62/935,035, filed Nov. 13, 2019 which is hereby incorporated by reference herein in its entirety.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in the ASCII text file:
206256-0024-00US SequenceListing.txt;
   created on Nov. 12, 2020, 1,945 bytes, is hereby incorporated by reference.

### STATEMENT REGARDING FEDERALLY SPONSORED RESEARCH OR DEVELOPMENT

This invention was made with government support under R01 HL123340 and R01 DK093668 awarded by the National Institutes of Health (NIH). The government has certain rights in the invention.

### BACKGROUND OF THE INVENTION

Treatment of complex inflammatory disorders often involves “step-up” approaches in which patients are initially prescribed mild therapies, and upon failure to demonstrate improvement, receive interventions of increasing intensity and risk. Multiple rounds of empiric testing and failure of treatments presents a substantial burden on the healthcare system that contributes to decreased quality of life, and can negatively impact the disease course. The promise of precision medicine is that certain features of the patient will predict responsiveness to therapies and circumvent the need for such trial and error approaches. However, biomarker analysis of blood or other tissue specimens has had only limited success. An alternative approach is to establish an ex vivo assay in which disease-related events are recreated with patient-derived material, and then subsequently applied to test drug responsiveness.

Allogeneic hematopoietic cell transplantation (allo-HCT) involving the transfer of bone marrow (BM), peripheral blood, or cord blood from a non-identical donor can be a life-saving procedure. When applied to treat malignancies such as myeloid leukemia, donor-derived T cells contribute to remission by attacking the tumor cells in recipients. However, in as many as 50% of transplant recipients, these alloreactive T cells attack healthy tissues and cause a multi-organ disorder termed graft-versus-host disease (GVHD) (Welniak et al., 2007, Annu Rev Immunol 25, 139-170; Jenq and van den Brink, 2010, Nat Rev Cancer 10, 213-221, Li et al., 2008, Expert Opin Pharmacother 9, 2305-2316). The gastrointestinal tract is one of the major target organs and accounts for much of the morbidity and mortality associated with GVHD (Ferrara et al., 2017, J Clin Invest 127, 2441-2451). Despite the high frequency of intestinal GVHD among allo-HCT recipients, few biomarkers and methods are currently available that predict intestinal involvement or response to treatment (Stellj es et al., 2008, Blood 111, 2909-2918; Rodriquez-Otero et al., 2012, Blood 119, 5909-5917; Major-Monfried et al., 2018, Blood 131, 2846-2855).

The autophagy gene ATG16L1 is protective during allo-HCT (Hubbard-Lucey et al., 2014, Immunity 41, 579-591; Matsuzawa-Ishimoto et al., 2017, J Exp Med 214, 3687-3705). A common polymorphism in ATG16L1 (ATG16L1^{T300A}) was initially identified as a susceptibility factor for the inflammatory bowel disease (IBD) Crohn's disease (Khor et al., 2011, Nature 474, 307-317). Both intestinal GVHD and Crohn's disease frequently involve the distal small intestine, but can involve any part of the gastrointestinal tract, and are characterized by overproduction of Th1 cytokines TNFα and IFNγ, epithelial barrier disruption, and alterations in the composition of the microbiota (Ferrara et al., 2017, J Clin Invest 127, 2441-2451; Khor et al., 2011, Nature 474, 307-317; Shono and van den Brink, 2018, Nat Rev Cancer 18, 283-295)(4, 10, 11). Based on these similarities between the two groups of disorders, the role of ATG16L1 in GVHD was examined and it was found that mice with reduced Atg16L1 expression were susceptible to GVHD in an animal model of allo-HCT, and that the ATG16L1^{T300A} variant was associated with increased transplant-related mortality in human allo-HCT recipients (Hubbard-Lucey et al., 2014, Immunity 41, 579-591). More recently, it was shown that mice with deletion of ATG16L1 in intestinal epithelial cells (IECs) displayed exacerbated GVHD following allo-HCT, indicating that Atg16L1 deletion in IECs is sufficient to confer increased susceptibility (Matsuzawa-Ishimoto et al., 2017, J Exp Med 214, 3687-3705).

Autophagy is a conserved pathway by which cellular material including organelles and long-lived proteins are degraded and recycled when they are sequestrated by double-membrane vesicles that fuse with endo-lysosomes (Galluzzi et al., 2017, EMBO J 36, 1811-1836; Matsuzawa-Ishimoto et al., 2018, Annu Rev Immunol 36, 73-101). Mice harboring IEC-specific deletions of ATG16L1 or other autophagy proteins display impaired viability of several epithelial lineages, including enterocytes, Paneth cells, and goblet cells (Burger et al., 2018, Cell Host Microbe 23, 177-190 e174; Adolph et al., 2013, Nature, 503(7475):272-276; Pott et al., 2018, Cell Host Microbe 23, 191-202 e194; Asano et al., 2017, Cell Rep 20, 1050-1060; Slowicka et al., 2019, Nat Commun 10, 1834). ATG16L1 inhibits a form of programmed necrosis termed necroptosis in murine intestinal organoids (Matsuzawa-Ishimoto et al., 2017, J Exp Med 214, 3687-3705; Aden et al., 2018, J Exp Med 215, 2868-2886), a self-renewing three-dimensional (3D) cell culture system in which IEC lineages are differentiated from primary epithelial stem cells (Sato et al., 2009, Nature 459, 262-265). Necroptosis can occur when activation of cytokine and death receptors induce the formation of a complex consisting of receptor interacting protein kinase RIPK3 (RIP3) and RIPK1 (RIP1) that mediates the recruitment and phosphorylation of the pore-forming molecule mixed lineage kinase domain-like (MLKL) (Pasparakis and Vandenabeele, 2015, Nature 517, 311-320). The role of autophagy proteins is cell type-dependent and can promote necroptosis in prostate tumor cells (Goodall et al., 2016, Dev Cell 37, 337-349; Tait et al., 2013, Cell Rep 5, 878-885; Lu et al., 2016, PLoS One 11, e0147792). Although evidence is presented that ATG16L1 prevents TNFα-induced cell death of intestinal organoids by mediating the autophagic removal of mitochondria that produce reactive oxygen species (ROS) (Matsuzawa-Ishimoto et al., 2017, J Exp Med 214, 3687-3705), how intracellular signaling is disrupted under these conditions is obscure. Additionally, the relevance to human disease requires further investigation.

Thus, there is a need in the art for improved individualized therapies that target affected tissues. The present invention satisfies this unmet need.

### SUMMARY OF THE INVENTION

In some embodiments, the invention relates to methods of treating or preventing a disease or disorder associated with immune response-mediated tissue injury in a subject in need thereof, the method comprising: identifying the subject as having an inactivating mutation in the Autophagy Related 16 Like 1 gene (ATG16L1), and administering to the subject at least one of an inhibitor of necroptosis and an inhibitor of interferon signaling.

In some embodiments, the inactivating mutation in ATG16L1 is a T300A mutation.

In some embodiments, the subject has been diagnosed with intestinal graft-versus-host disease (GVHD), inflammatory bowel disease (IBD), Crohn's disease (CD), ulcerative colitis (UC), pouchitis, irritable bowel syndrome (MS), infectious and non-infectious gastroenteritis, autoimmunity associated with cancer immunotherapy, gastrointestinal cancer, or radiation enteritis.

In some embodiments, the inhibitor is a chemical compound, a protein, a peptide, a peptidomimetic, an antibody, a ribozyme, a small molecule chemical compound, a nucleic acid, a vector, or an antisense nucleic acid molecule.

In some embodiments, the inhibitor is an inhibitor of at least one of RIPK1, RIPK3, MLKL and JAK/STAT.

In some embodiments, the inhibitor is a RIPK1 inhibitor. In some embodiments, the inhibitor is a Necrostatin, Vorinostat, 1-Benzyl-1H-pyrazole derivatives, aminoisoquinolines, PN10, Cpd27, GSK′840, GSK′843, GSK′872, Curcumin, tozasertib, ponatinib, pazopanib, GSK2982772, DNL747, or a small molecule inhibitor or an analog or derivative thereof.

In some embodiments, the inhibitor is a RIPK3 inhibitor. In some embodiments, the inhibitor is GSK′840, GSK′843, GSK′872, Ganoderma lucidium Mycelia, Kongensin A, Celastrol, ponatinib, HS-1371, or dabrafenib or analogs or derivatives thereof.

In some embodiments, the inhibitor is a MLKL inhibitor. In some embodiments, the inhibitor is ponatinib, pazopanib, necrosulphonamide, Compound 1, Celastrol, or TC13172, or analogs or derivatives thereof.

In some embodiments, the inhibitor is a JAK/STAT inhibitor. In some embodiments, the inhibitor is tofacitinib, ruxolitinib, peficitinib, filgotinib, solcitinib, upadacitinib, baricitinib, itacitinib, SHR0302, PF04965842, or decernotinib or analogs or derivatives thereof.

In some embodiments, the inhibitor is a necroptosis inhibitor. In some embodiments, the inhibitor is furo[2,3-d]pyrimidine, pyrrolo[2,3-b]pyridines, IM-54, a NecroX analog, GSK2982772, Terminalia Chebula, Naringenin, a small molecule necroptosis inhibitor, a tricyclic necrostatin compound, a heterocyclic inhibitor of necroptosis, a spiroquinoxaline derivative, tofacitinib, ruxolitinib, peficitinib, filgotinib, solcitinib, or upadacitinib, or analogs or derivatives thereof.

In some embodiments, the invention relates to methods for preparing an intestinal organoid-immune cell co-culture, wherein the method comprises culturing small intestinal and colonic crypt cells in contact with an extracellular matrix to obtain an intestinal organoid; removing said extracellular matrix from said intestinal organoids; preparing an immune cell suspension comprising stimulated immune cells; mixing the immune cell suspension comprising stimulated immune cells with the intestinal organoids; and resuspending the intestinal organoid-immune cell co-culture in an extracellular matrix.

In some embodiments, the small intestinal and colonic crypt cells are cultured in a medium comprising mEGF, mNoggin and mR-Spondin 1.

In some embodiments, the immune cells are T cells.

In some embodiments, the small intestinal and colonic crypt cells and immune cells are obtained from the same subject. In some embodiments, the small intestinal and colonic crypt cells and immune cells are human cells.

In some embodiments, the invention relates to an intestinal organoid culture obtained by the method of culturing small intestinal and colonic crypt cells in contact with an extracellular matrix to obtain an intestinal organoid; removing said extracellular matrix from said intestinal organoids and re-suspending the organoids in a medium. In one embodiment, the medium comprises at least one additional agent. In some embodiments, the additional agent is an immune cell. In some embodiments, the additional agent is an inflammatory cytokine.

In some embodiments, the invention relates to methods for testing a therapeutic agent, wherein the method comprises contacting an intestinal organoid-culture or co-culture with one or more candidate agents, detecting the presence or absence of one or more change in the intestinal organoid culture or co-culture that is indicative of therapeutic efficacy, and identifying a candidate agent as a therapeutic agent if the presence or absence of one or more of said changes in the intestinal organoid culture or co-culture is detected.

In some embodiments, the said change in the intestinal organoid cell culture or co-culture is an increase in cell viability, organoid size, morphology, quantification of epithelial subsets, cell proliferation, transcriptome protein levels or post-translational modifications of proteins, metabolism, production of soluble factors or any combination thereof of the intestinal organoid cells as compared to a comparator control.

In some embodiments, the therapeutic agent is suitable for the treatment of a disease or disorder associated with immune response-mediated tissue injury.

In some embodiments, the disease or disorder associated with immune response-mediated tissue injury is GVHD, IBD, CD, UC, pouchitis, IBS, infectious or non-infectious gastroenteritis, autoimmunity associated with cancer immunotherapy, gastrointestinal cancer, or radiation enteritis.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A through FIG. 1G depict exemplary experimental results demonstrating that ATG16L1 in the intestinal epithelium protects against lethal GVHD mediated by RIP1 and RIP3. FIG. 1A depicts a schematic representation of the preclinical GVHD model. CPA; Cyclophosphamide. HCT; Hematopoietic Cell Transplantation. FIG. 1B depicts the survival of Atg16L1^{f/f} (f/f) and Atg16L1^{ΔIEC} (ΔIEC) mice receiving a chemotherapy conditioning regimen and transplanted with 5×10⁶ T cell-depleted BM cells with or without 4×10⁶ splenic T cells from donor LP/J mice. n=9 (f/f, BM only), 9 (ΔIEC, BM only), 9 (f/f, BM+ T cells), and 7 (ΔIEC, BM+ T cells). FIG. 1C depicts the clinical disease scores (see Materials and Methods) evaluated every 7 days after allo-HCT in FIG. 1B. FIG. 1D depicts the survival of chemotherapy-pretreated Atg16L1^{f/f}×Rip3^{-/-} (f/f Rip3^{-/-}) and Atg16L1^{ΔIEC}×Rip3^{-/-} (ΔIEC Rip3^{-/-}) mice transplanted with 5×10⁶ T cell-depleted BM cells with or without 4×10⁶ splenic T cells from donor LP/J mice. n=11 (f/f Rip3^{-/-}, BM only), 10 (ΔIEC Rip3^{-/-} BM only), 9 (f/f Rip3^{-/-}, BM+ T cells), and 8 (ΔIEC Rip3^{-/-}, BM+ T cells). FIG. 1E depicts the clinical disease scores evaluated every 7 days after allo-HCT in FIG. 1D. FIG. 1F depicts the survival of chemotherapy-pretreated Atg16L1^{f/f} (f/f) and Atg16L1^{ΔIEC} (ΔIEC) mice which received GSK547 or control chow and were transplanted with 5×10⁶ T cell-depleted BM cells with 4×10⁶ splenic T cells from donor LP/J mice. n=8 (f/f, control), 8 (ΔIEC, control), 8 (f/f, GSK547), and 8 (ΔIEC, GSK547). GSK547 was started 10 days before allo-HCT, and continued until the end of the study. FIG. 1G depicts the clinical disease scores evaluated every 7 days after allo-HCT. Data points in FIG. 1B, FIG. 1D, and FIG. 1F represent individual mice, and data points in FIG. 1C, FIG. 1E, and FIG. 1G are mean of clinical scores of viable mice. Bars represent means±SEM, and survival data in FIG. 1B, FIG. 1D, and FIG. 1F are combined results of 2 experiments performed independently. FIG. 1B, FIG. 1D, and FIG. 1F were analyzed with the Mantel-Cox log rank test. In FIG. 1C, FIG. 1E, and FIG. 1G, AUC was determined for each mouse and difference between groups was analyzed using ANOVA with Tukey's multiple comparison test. *P<0.05, **P<0.01, ****P<0.0001.

FIG. 2A through FIG. 2C depict exemplary experimental results demonstrating additional characterization of mice deficient in ATG16L1 in the epithelium after allo-HCT. FIG. 2A depicts complete blood counts of B6 mice receiving a chemotherapy conditioning regimen (chemo) as in FIG. 1A. n=3 each condition. FIG. 2B and FIG. 2C depict mice receiving BM and T cells from donor LP/J mice as in FIG. 1B were sacrificed on day 28 post allo-HCT, and analyzed for flow cytometric analysis of indicated cells in the lamina propria of the small intestine (FIG. 2B) and colon (FIG. 2C). n=11 (Atg16L1^{f/f}; f/f), 12 (Atg16L1^{ΔIEC} ΔIEC). Data points represent individual mice. Bars represent means±SEM, and at least 2 independent experiments were performed. Data were analyzed by Student's t test. *P<0.05, **P<0.01.

FIG. 3A through FIG. 3D depict exemplary experimental results demonstrating that ATG16L1-Deficiency Prevents Intestinal GVHD by Inhibiting Epithelial Necroptosis. Mice receiving BM and T cells from donor LP/J mice as in FIG. 1 were sacrificed on day 28 post allo-HCT, and analyzed for signs of intestinal GVHD. n=11 (Atg16L1^{f/f}; f/f), 12 (Atg16L1^{ΔIEC} ΔIEC), 8 (Atg16L1^{f/f}×Rip3^{-/-}; f/f Rip3^{-/-}) and 8 (Atg16L1^{ΔIEC}×Rip3^{-/-}; ΔIEC Rip3^{-/-}). FIG. 3A depicts colon length. FIG. 3B depicts the pathology score of small intestine (SI), colon, liver, and skin. FIG. 3C depicts representative images and FIG. 3D depicts the quantification of H&E, periodic acid-Schiff (PAS)/Alcian blue, TUNEL, and cleaved-caspase3 staining. Arrowheads indicate Paneth cells or IECs positive for the indicated markers. Scale bars represent 10 μm in H&E, TUNEL, and Cleaved-caspase3, and 100 μm in PAS/Alcian blue. At least 50 crypt-villus units were quantified per mouse. Data points in FIG. 3A, FIG. 3B, and FIG. 3D represent individual mice. Bars represent means±SEM, and at least 2 independent experiments were performed. Data were analyzed using ANOVA with Tukey's multiple comparison test. *P<0.05, **P<0.01, ***P<0.001, ****P<0.0001.

FIG. 4A through FIG. 4C depict exemplary experimental results demonstrating that deletion of Atg16L1 in recipient intestinal epithelial tissue worsens intestinal GVHD following Allo-HCT. Mice receiving BM and T cells from donor LP/J mice as in FIG. 1 were sacrificed on day 28 post allo-HCT, and analyzed for signs of intestinal GVHD. n=11 (Atg16L1^{f/f}; f/f), 12 (Atg16L1^{ΔIEC}; ΔIEC), 8 (Atg16L1^{f/f}×Rip3^{-/-}; f/f Rip3^{-/-}) and 8 (Atg16L1^{ΔIEC}×Rip3^{-/-}; ΔIEC Rip3^{-/-}). FIG. 4A depicts representative images and FIG. 4B depicts quantification of TUNEL and Cleaved-caspase3 staining of colon. Scale bars represent 10 μm. At least 50 crypts were quantified per mouse. FIG. 4C depicts colony forming units (CFU) of bacteria present in spleen. Data points in FIG. 4B and FIG. 4C represent individual mice. Bars represent means±SEM, and at least 2 independent experiments were performed. Data were analyzed using ANOVA with Tukey's multiple comparison test. *P<0.05.

FIG. 5A and FIG. 5B depict exemplary experimental results demonstrating deletion of Atg16L1 in Intestinal Epithelial Cells Decreases the Surface MEW Class I. FIG. 5A depicts the schematic representation of an animal ex vivo intestinal GVHD model. FIG. 5B depicts flow cytometric analysis of MEW class I (MHC-I) in small intestinal organoids from Atg16L1^{f/f} (f/f) and Atg16L1^{ΔIEC} (ΔIEC) mice at day 3. Data points in FIG. 5B represent individual organoids sample. Bars represent means±SEM, and at least 2 independent experiments were performed (n=3). Data were analyzed by Student's t test. **P<0.01.

FIG. 6A through FIG. 6G depict exemplary experimental results demonstrating that allogeneic CD8⁺ T cells induce cell death in intestinal organoids with autophagy gene mutations. Representative images (FIG. 6A), viability (FIG. 6B) and size (FIG. 6C) of small intestinal organoids from B6-background Atg16L1^{f/f} (f/f) and Atg16L1^{ΔIEC} (ΔIEC) mice co-cultured for 48 hours with 1×10⁵ splenic T cells separately harvested from B6, B10.BR, and LP/J mice. n=3 mice each. Arrowheads indicate dead organoids. FIG. 6D depicts the viability of organoids from B6-background Atg4B and Atg16L1^{T316A} mice co-cultured for 48 hours with 1×10⁵ splenic T cells separately harvested from B10.BR mice. n=3 mice each. FIG. 6E depicts the viability of small intestinal organoids from f/f and ΔIEC mice co-cultured for 48 hours with FACS-sorted 1×10⁵ CD4⁺ or 7×10 4 CD8⁺ T cells from B10.BR mice. n=3 mice each. FIG. 6F and FIG. 6G depict representative images (FIG. 6F) and number (FIG. 6G) of T cells associated with organoid. At least 50 organoids were analyzed per group. T cells were stained with Cell Bright Green (green) before co-culture, and PI (red) was added into the culture medium at the beginning to stain dead organoids/T cells. Scale bars represent 400 μm in FIG. 6A and 25 μm in FIG. 6F. n=3 mice each. Data points in FIG. 6B, FIG. 6D and FIG. 6E are mean of technical replicates, and data points in FIG. 6C and FIG. 6G represent individual organoids. Bars represent means±SEM, and at least 2 independent experiments were performed. Data were analyzed using ANOVA with Tukey's multiple comparison test. *P<0.05, **P<0.01, ***P<0.001, ****P<0.0001.

FIG. 7A through FIG. 7H depict exemplary experimental results demonstrating that allogeneic T cells induce TNFα-mediated necroptosis in intestinal organoids. FIG. 7A depicts the fold change of indicated cytokines in culture supernatants from FIG. 6B. Each value is normalized to non-stimulated samples. n=3 mice each. FIG. 7B depicts the viability of small intestinal organoids treated ±anti-TNFα and/or anti-IFNγ antibody and co-cultured with B10.BR T cells for 48 hours. n=3 mice each. FIG. 7C and FIG. 7F depict representative images of co-cultured small intestinal (FIG. 7C) and colonic (FIG. 7F) organoids. Dead organoids were pointed with arrowheads. Scale bars represent 100 μm. FIG. 7D and FIG. 7G depict the viability of small intestinal (FIG. 7D) and colonic (FIG. 7G) organoids from B6-background Atg16L1^{f/f} (f/f), Atg16L1^{ΔIEC} (ΔIEC), Atg16L1^{f/f}×Rip3^{-/-} (f/f Atg16L1^{ΔIEC}×Rip3^{-/-} (ΔIEC Rip3^{-/-}) mice co-cultured for 48 hours with B10.BR T cells. n=3 mice each. FIG. 7E and FIG. 7H depict the size of co-cultured small intestinal (FIG. 7E) and colonic (FIG. 7H) organoids in (FIG. 7C) and (FIG. 7F). n=3 mice each. Data points in (FIG. 7A), (FIG. 7B), (FIG. 7D), and (FIG. 7G) are mean of technical replicates, and data points in (FIG. 7E) and (FIG. 7H) represent individual organoids. Bars represent means±SEM, and at least 2 independent experiments were performed. Data were analyzed using ANOVA with Tukey's multiple comparison test. *P<0.05, **P<0.01, ***P<0.001, ****P<0.0001.

FIG. 8 depicts exemplary experimental results demonstrating the quantification of cytokines in culture supernatants from the Ex Vivo GVHD model. The fold change of indicated cytokines in culture supernatants from FIG. 6B. Each value is normalized to non-stimulated samples. Data points are the mean of technical replicates. Bars represent means±SEM, and at least 2 independent experiments were performed. Data were analyzed using ANOVA with Tukey's multiple comparison test. *P<0.05, **P<0.01, ***P<0.001, ****P<0.0001.

FIG. 9A through FIG. 9F depict exemplary experimental results demonstrating loss of viability in ATG16L1-deficient intestinal organoids is associated with an interferon signature. FIG. 9A depict unsupervised clustering based on expression of most variable genes by genotype and treatment with 20 ng/ml TNFα for 2 hours. n=4 replicates per group, each replicate was derived from separate mice. FIG. 9B depicts a heatmap of genes with 2× fold change in Atg16L1^{ΔIEC} (ΔIEC) over Atg16L1^{f/f} (f/f) organoids. Each color indicates z-score. Interferon stimulated genes (ISGs) were exaggerated as red and bold. FIG. 9C depicts a pathway analysis of genes differentially expressed between f/f and ΔIEC naïve organoids. FIG. 9D depicts a quantitative RT-PCR measurement of indicated ISG expression normalized to actb in small intestinal organoids from B6 mice±100 nM Ruxolitinib at day 3. n=3 mice each. FIG. 9E depicts the viability of small intestinal organoids stimulated with 20 ng/ml TNFα and/or 100 nM Ruxolitinib for 48 hours. n=3 mice each. FIG. 9F depicts a western blot analysis of necroptosis-related proteins in day 3. f/f and ΔIEC organoids cultured±100 nM Ruxolitinib were treated with 20 ng/ml TNFα for 2 hours. Blots are representative of at least 2 independent repeats. Data points in (FIG. 9D) and (FIG. 9E) are mean of technical replicates. Bars represent means±SEM, and at least 2 independent experiments were performed. Data in (FIG. 9E) were analyzed using ANOVA with Tukey's multiple comparison test. ***P<0.001, ****P<0.0001.

FIG. 10A through FIG. 10B depict exemplary experimental results demonstrating TNFα-induced gene expression changes in organoids with or without ATG16L1 deletion. FIG. 10A depicts Venn diagrams of genes upregulated 2-fold or greater by TNFα in Atg16L1^{f/f} (f/f) and ΔIEC organoids. FIG. 10B depicts a pathway analysis of the 175 genes upregulated in TNFα-treated Atg16L1^{ΔIEC} (ΔIEC) organoids compared with TNFα-treated Atg16L1^{f/f} organoids.

FIG. 11A through FIG. 11E depict exemplary experimental results demonstrating the establishment and characterization of a human organoid-allogeneic T cells co-culture model. FIG. 11A depicts a schematic representation of an ex vivo GVHD model using human tissues. FIG. 11B depicts representative images of frozen human organoids at day 0 (left) and 3 (right) after thawing. FIG. 11C depicts the frequency of viable, CD3⁺, and CD4⁺/CD8α⁺ lymphocytes in thawed human allogeneic T cells before co-culture (after T-cell sorting). FIG. 11D depicts the proportions of organoids which are susceptible to either TNFα or allogeneic T cells. Low; not statistically susceptible (P>0.05). Moderate; lethality <50%. High; lethality >50%. FIG. 11E depicts Venn diagram of organoids which are highly susceptible to either TNFα or allogeneic T cells. At least 2 independent experiments were performed.

FIG. 12A through FIG. 12D depict exemplary experimental results demonstrating the development of an ex vivo intestinal GVHD model using human intestinal organoids and peripheral T cells. FIG. 12A depicts representative images of human small intestinal organoids co-cultured for 8 hours with syngeneic (syn) or allogeneic (allo) human T cells. Sorted T cells were stained with Cell Bright Green before co-culture, and PI was added into the culture medium at the beginning to stain dead organoids. Scale bars represent 25 μm. FIG. 12B and FIG. 12C depict the viability of human small intestinal organoids from 20 different patients at 48 hours after stimulation with 50 ng/ml TNFα or post co-culture with allogeneic and syngeneic T cells (FIG. 12B) or with only allogeneic T cells (FIG. 12C). FIG. 12D depicts the viability of human colonic organoids from 4 different patients at 48 hours after stimulation with 50 ng/ml TNFα or post co-culture with allogeneic and/or syngeneic T cells. At least 2 independent experiments were performed. *p<0.05, **p<0.01, ***p<0.001, ****p<0.0001.

FIG. 13A through FIG. 13D depict exemplary experimental results demonstrating the intestinal organoids derived from ATG16L1T300A homozygous individuals display heightened susceptibility to TNFα and allogeneic T cells. FIG. 13A depicts the proportion of human small intestinal organoids from FIGS. 12B and 12C which were susceptible (displayed >50% lethality) to recombinant TNFα (left) or allogeneic T cells (right). N=14 (Nonrisk) and 6 (T300A/T300A). Statistical significance was validated with Fisher's exact test. FIG. 13B depicts a combined organoid viability in (A). N=14 (Non-risk) and 6 (T300A/T300A). FIG. 13C and FIG. 13D depict representative images (FIG. 13C) and viability (FIG. 13D) of human small intestinal organoids stimulated with or without 50 ng/ml TNFα, 100 nM Ruxolitinib, 1 μM GSK547, 20 μM Necrostatin-1s (Nec-1s), and 2 μM necrosulfonamide (NSA) for 48 hours. Scale bars represent 400 μm. Data points in FIG. 13B represent an average viability of individual organoids in FIG. 12, and data points in FIG. 13C are mean of technical replicates. At least 2 independent experiments were performed. ***p<0.001, ****p<0.0001.

FIG. 14A through FIG. 14D depict exemplary experimental results demonstrating the susceptibility of TNFα in organoids derived from subjects with ulcerative colitis. Viability over time of organoids from 18 ulcerative colitis (UC) patients (9 naive, 4 responsive, and 5 refractory to anti-TNFα) was measured by microscopy following treatment with 20 or 40 ng/ml recombinant human TNFα. Organoids were also treated with 10 or 20 ng/ml human interferon gamma (IFNγ) as a control cytokine expected to be toxic. (FIG. 14A) Organoids derived from anti-TNF-responsive patients were susceptible TNFα (n=4). (FIG. 14B) Organoids derived from anti-TNF-refractory patients were resistant to TNFα (n=5). (FIG. 14C and FIG. 14D) Organoids derived from anti-TNF-naive patients could be divided into either (FIG. 14C) susceptible (n=6) or (FIG. 14D) resistant groups. These results indicate that organoids from UC patients can be segregated based on their sensitivity to cytokines, which is reflective of the patient's clinical responsiveness to treatments. Bars represent mean±SD. Carrier protein, PBS containing 0.1% (w/v) BSA. ns, not significant; *, P<0.05 by paired t test.

FIG. 15A through FIG. 15E depict exemplary experimental results demonstrating that IL-17 treatment identified responsive and unresponsive organoids. (FIG. 15A) Intestinal organoids from small intestinal biopsies procured from nine individuals were differentiated in the presence of 10 ng/ml of the cytokine IL-17A. Four of the nine organoids (R1-R4) responded to IL-17A treatment by converting from cystic morphology to displaying buds, a sign of enhanced differentiation of secretory epithelial cells. In contrast, five out of the nine were unresponsive (UR1-UR5) and displayed similar morphology when comparing IL-17A treated and control carrier protein only. Unresponsive organoids were characterized by budding in the absence of IL-17A. Scale bar=400 μM. (FIG. 15B) quantitative RT-PCR (qPCR) analysis indicates that organoids identified as responsive above display enhanced expression of the indicated genes associated with secretory epithelial cells: LYZ (Paneth cells), ATOH1 (secretory lineage commitment), MUC2 and CLCA/(goblet cells), and CHGA (enteroendocrine cells). (FIG. 15C) qPCR analysis of unresponsive lines indicates that these lineage markers are not altered in these organoids. (FIG. 15D) Responsive lines are characterized by higher expression of the receptor for IL-17 (IL-17RA). (FIG. 15E) Gene expression results were validated by staining sections of representative responsive organoids (R3 and R4) for MUC2 and CHGA at the protein level with antibodies and visualizing by fluorescent microscopy on day 8 post IL-17A treatment. These results indicate that organoids display distinct morphologies, which can be further affected by the immune effector molecule IL-17A. Organoids that are sensitive to IL-17A respond through enhanced differentiation of secretory cell lineage. Gene expression values represent fold change and are normalized to actin. Bars represent mean±SD. ns, not significant; *, P<0.05; **, P<0.01; ***, P<0.001; ****, P<0.0001 by paired t test.

### DETAILED DESCRIPTION

The invention is based, in part, on experiments demonstrating that intestinal GVHD in mice deficient in the Autophagy Related 16 Like 1 gene (Atg16L1), an autophagy gene that is polymorphic in humans, is reversed by inhibiting necroptosis. Further, the invention is based, in part, on the demonstration that co-cultured allogeneic T cells kill Atg16L1 mutant intestinal organoids from mice, which was associated with an aberrant epithelial interferon signature. Therefore, in one embodiment, the invention provides compositions and methods for inhibiting necroptosis or interferon signaling to treat diseases and disorders associated with immune response-mediated tissue injury in individuals harboring an inactivating mutation in Atg16L1.

In some embodiments, the disease or disorder is intestinal graft-versus-host disease (GVHD), inflammatory bowel disease (IBD), Crohn's disease (CD), ulcerative colitis (UC), pouchitis, irritable bowel syndrome (IBS), infectious and non-infectious gastroenteritis, autoimmunity associated with cancer immunotherapy, gastrointestinal cancer (e.g., esophageal, colorectal, small intestine, oral, and gastric cancer), or radiation enteritis.

In another aspect, the invention is based, in part, on the development of an ex vivo platform to evaluate individual-specific responses to agents of interest, such as cytokines, immune cells, and potential therapeutic agents. For example, in one embodiment, the platform recreates genetic susceptibility to T cell-mediated damage. In one embodiment, the ex vivo platform comprises epithelial or intestinal organoids of a subject. In one embodiment, the ex vivo platform comprises a co-culture of organoids and immune cells. For example, in one embodiment, the ex vivo platform comprises a co-culture of organoids and immune cells, each of or derived from the same subject. In one embodiment, the ex vivo platform comprises organoids, immune cells, or both from a subject having an inactivating mutation in Atg16L1.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “anti-tumor effect” as used herein, refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

“Co-culture” refers to two or more cell types maintained together in the same culture chamber, such as a dish, tube, container, or the like. In some embodiments, the two or more cell types are maintained in conditions suitable for their mutual function or in conditions for their mutual interaction. In the context of the present disclosure, an “organoid co-culture” relates to an epithelial organoid, as defined elsewhere, in culture with a non-epithelial cell type, specifically an immune cell type. In some embodiments, cell types in co-culture exhibit a structural, biochemical and/or phenomenological association that they do not exhibit in isolation. In some embodiments, cell types in co-culture mimic the structural, biochemical and/or phenomenological association observed between the cell types in vivo.

“Immune disease” refers to any disorder of the immune system. Immune diseases include autoimmune diseases (in which the immune system erroneously acts upon self-components) and immune-mediated diseases (in which the immune system exhibits excessive function).

“Immunotherapy” refers to any medical intervention that induces, suppresses or enhances the immune system of a patient for the treatment of a disease. In some embodiments, immunotherapies activate a patient's innate and/or adaptive immune responses (e.g. T cells) to more effectively target and remove a pathogen or cure a disease, such as cancer or an immune disease.

“Intestine” and “intestinal” refer to the gastrointestinal tract, including the mouth, oral cavity, esophagus, stomach, large intestine, small intestine, rectum, and anus.

“Organoid” refers to a cellular structure obtained by expansion of adult (post-embryonic) epithelial stem cells, consisting of tissue-specific cell types that self-organize through cell sorting and spatially restricted lineage commitment.

As used herein, the term “conservative sequence modifications” is intended to refer to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody or antibody fragment of the invention can be replaced with other amino acid residues from the same side chain family and the altered antibody or antibody fragment can be tested for the ability to bind CD123 using the functional assays described herein.

Unless otherwise specified, a “nucleotide sequence encoding an amino acid sequence” includes all nucleotide sequences that are degenerate versions of each other and that encode the same amino acid sequence. Nucleotide sequences that encode proteins or a RNA may also include introns to the extent that the nucleotide sequence encoding the protein may in some version contain an intron(s).

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its regulatory sequences.

A “transfer vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, polylysine compounds, liposomes, and the like. Examples of viral transfer vectors include, but are not limited to, adenoviral vectors, adeno-associated virus vectors, retroviral vectors, lentiviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

A “lentiviral vector” is a vector derived from at least a portion of a lentivirus genome, including especially a self-inactivating lentiviral vector as provided in Milone et al., Mol. Ther. 17(8): 1453-1464 (2009). Other Examples or lentivirus vectors that may be used in the clinic as an alternative to the pELPS vector, include but not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “operably linked” or alternatively “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, where necessary to join two protein coding regions, are in the same reading frame.

“Parenteral” administration of an immunogenic composition includes, e.g., subcutaneous (s.c.), intravenous (i.v.), intramuscular (i.m.), or intrasternal injection, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals including human).

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

By the term “synthetic” as it refers to a nucleic acid or polypeptide, including an antibody, is meant a nucleic acid, polypeptide, including an antibody, which has been generated by a mechanism not found naturally within a cell. In some instances, the term “synthetic” may include and therefore overlap with the term “recombinant” and in other instances, the term “synthetic” means that the nucleic acid, polypeptide, including an antibody, has been generated by purely chemical or other means.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses.

By the term “specifically binds,” as used herein, is meant an antibody or antigen binding fragment thereof, or a ligand, which recognizes and binds with a cognate binding partner present in a sample, but which antibody, antigen binding fragment thereof or ligand does not substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

In one aspect, the present invention provides methods to treat a disease or disorder associated with immune response-mediated tissue injury in a subject in need thereof. In one embodiment, the method of the present invention comprises administering an inhibitor of necroptosis signaling to a subject identified as having an inactivating mutation in the Autophagy Related 16 Like 1 gene (ATG16L1). In one embodiment, the inactivating ATG16L1 mutation is a T300A mutation.

The methods of the present invention can be used to treat or prevent any type of disease or disorder associated with an inactivating mutation in ATG16L1 including, but not limited to cancer and autoimmune and disorders. Diseases and disorders that can be treated by the disclosed methods include, but are not limited to, of intestinal graft-versus-host disease (GVHD), inflammatory bowel disease (IBD), Crohn's disease (CD), ulcerative colitis (UC), pouchitis, irritable bowel syndrome (IBS), infectious and non-infectious gastroenteritis, autoimmunity associated with cancer immunotherapy, gastrointestinal cancer, including, but not limited to, esophageal, colorectal, small intestine, oral, and gastric cancer, and radiation enteritis.

### Inhibitors of Necroptosis and Interferon Signaling

In various embodiments, the present invention includes compositions for inhibiting necroptosis or interferon signaling for use in methods of treating diseases and disorders associated with an inactivating mutation in ATG16L1 in a subject. In one embodiment, the inhibitor is an inhibitor of at least one of RIPK1, RIPK3, MLKL and JAK/STAT.

In one embodiment, the inhibitor of the invention decreases the amount of at least one of RIPK1, RIPK3, MLKL or JAK/STAT polypeptide, the amount of at least one of RIPK1, RIPK3, MLKL or JAK/STAT mRNA, the activity of at least one of RIPK1, RIPK3, MLKL or JAK/STAT, or a combination thereof.

It will be understood by one skilled in the art, based upon the disclosure provided herein, that a decrease in the level of polypeptide encompasses the decrease in the expression, including transcription, translation, or both. The skilled artisan will also appreciate, once armed with the teachings of the present invention, that a decrease in the level of polypeptide includes a decrease in the polypeptide activity. Thus, decrease in the level or activity of a polypeptide includes, but is not limited to, decreasing the amount of polypeptide, and decreasing transcription, translation, or both, of a nucleic acid encoding the polypeptide; and it also includes decreasing any activity of the polypeptide as well.

In one embodiment, the invention provides a generic concept for inhibiting at least one of RIPK1, RIPK3, MLKL or JAK/STAT polypeptide. In one embodiment, the composition of the invention comprises an inhibitor of at least one of RIPK1, RIPK3, MLKL or JAK/STAT polypeptide. In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

One skilled in the art will appreciate, based on the disclosure provided herein, that one way to decrease the mRNA and/or protein levels of at least one of RIPK1, RIPK3, MLKL or JAK/STAT polypeptide in a cell is by reducing or inhibiting expression of the nucleic acid encoding at least one of RIPK1, RIPK3, MLKL or JAK/STAT polypeptide. Thus, the protein level of at least one of RIPK1, RIPK3, MLKL or JAK/STAT polypeptide in a cell can also be decreased using a molecule or compound that inhibits or reduces gene expression such as, for example, siRNA, an antisense molecule or a ribozyme. However, the invention should not be limited to these examples.

In one embodiment, siRNA is used to decrease the level of at least one of RIPK1, RIPK3, MLKL or JAK/STAT polypeptide. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of RIPK1, RIPK3, MLKL or JAK/STAT at the protein level using RNAi technology.

In other related aspects, the invention includes an isolated nucleic acid encoding an inhibitor, wherein an inhibitor such as an siRNA or antisense molecule, inhibits RIPK1, RIPK3, MLKL or JAK/STAT, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York) and as described elsewhere herein. In another aspect of the invention, necroptosis or interferon signaling or a regulator thereof, can be inhibited by way of inactivating and/or sequestering one or more of RIPK1, RIPK3, MLKL or JAK/STAT, or a regulator thereof. As such, inhibiting the effects of RIPK1, RIPK3, MLKL or JAK/STAT can be accomplished by using a transdominant negative mutant.

In another aspect, the invention includes a vector comprising an siRNA or antisense polynucleotide. Preferably, the siRNA or antisense polynucleotide is capable of inhibiting the expression of RIPK1, RIPK3, MLKL or JAK/STAT. The incorporation of a desired polynucleotide into a vector and the choice of vectors is well-known in the art.

The siRNA or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

In some embodiments of the invention, an antisense nucleic acid sequence which is expressed by a plasmid vector is used to inhibit RIPK1, RIPK3, MLKL or JAK/STAT. In some embodiments, the antisense expressing vector is administered to a mammalian cell or the mammal itself, thereby causing reduced endogenous expression of RIPK1, RIPK3, MLKL or JAK/STAT.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit necroptosis or interferon signaling. Ribozymes useful for inhibiting the expression of a target molecule may be designed by incorporating target sequences into the basic ribozyme structure which are complementary, for example, to the mRNA sequence of RIPK1, RIPK3, MLKL or JAK/STAT of the present invention. Ribozymes targeting RIPK1, RIPK3, MLKL or JAK/STAT may be synthesized using commercially available reagents (Applied Biosystems, Inc., Foster City, CA) or they may be genetically expressed from DNA encoding them.

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core—building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In another aspect of the invention, RIPK1, RIPK3, MLKL or JAK/STAT can be inhibited by way of inactivating and/or sequestering the polypeptide. As such, inhibiting the effects of RIPK1, RIPK3, MLKL or JAK/STAT can be accomplished by using a transdominant negative mutant. Alternatively, an antibody specific for RIPK1, RIPK3, MLKL or JAK/STAT (e.g., an antagonist to RIPK1, RIPK3, MLKL or JAK/STAT) may be used. In one embodiment, the antagonist is a protein and/or compound having the desirable property of interacting with a binding partner of RIPK1, RIPK3, MLKL or JAK/STAT and thereby competing with the corresponding protein. In another embodiment, the antagonist is a protein and/or compound having the desirable property of interacting with RIPK1, RIPK3, MLKL or JAK/STAT and thereby sequestering RIPK1, RIPK3, MLKL or JAK/STAT.

As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, NY). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic affinity cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, NY).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, NY) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

Exemplary inhibitors of RIPK1 include, but are not limited to, Necrostatins including, but not limited to, Nec-1, Nec-2, Nec-3, Nec-1s, 7-C1-Nec-1, 7-Cl—O-Nec-1, R-7-C1-O-Nec-1, Nec-5, and Nec-7, Vorinostat, 1-Benzyl-1H-pyrazole derivatives, aminoisoquinolines, PN10, Cpd27, GSK′840, GSK′843, GSK′872, Curcumin, Tozasertib (VX-680 and MK-0457), ponatinib, pazopanib, GSK2982772, DNL747 and small molecule inhibitors and analogs and derivatives thereof.

Exemplary inhibitors of RIPK3 include, but are not limited to, GSK′840, GSK′843, GSK′872, Ganoderma lucidium Mycelia, Kongensin A, Celastrol, ponatinib, HS-1371, dabrafenib and analogs and derivatives thereof.

Exemplary inhibitors of MLKL include, but are not limited to, ponatinib, pazopanib, necrosulphonamide, Compound 1, TC13172 and Celastrol and analogs and derivatives thereof.

Exemplary inhibitors of JAK/STAT include, but are not limited to, tofacitinib, ruxolitinib, peficitinib, filgotinib, solcitinib, baricitinib, itacitinib, SHR0302, PF04965842, decernotinib and upadacitinib, and analogs and derivatives thereof.

Exemplary necroptosis inhibitors include, but are not limited to, furo[2,3-d]pyrimidines, pyrrolo[2,3-b]pyridines, IM-54, a NecroX analog (NecroX-1, NecroX-2, NecroX-5, and NecroX-7), GSK2982772, Terminalia Chebula, Naringenin, a small molecule necroptosis inhibitor, a tricyclic necrostatin compound, a heterocyclic inhibitor of necroptosis, a spiroquinoxaline derivative, tofacitinib, ruxolitinib, peficitinib, filgotinib, solcitinib, and upadacitinib, and analogs and derivatives thereof.

Combination of Inhibitors

In one embodiment, the invention relates to a composition comprising a combination of inhibitors, and the use of a combination of inhibitors for the treatment of a disease or disorder associated with immune response-mediated tissue injury. In some embodiments, the combination of inhibitors inhibits a combination of necroptosis and interferon signaling. In some embodiments, the combination of inhibitors inhibits a combination of RIPK1, RIPK3, MLKL and JAK/STAT signaling. In some embodiments, the combination of inhibitors inhibits a combination of tumor necrosis factor alpha (TNF-α), and interferon-gamma (IFN-γ). Exemplary TNF-α inhibitors that can be administered according to the methods of the invention include, but are not limited to, anti-TNF-α antibodies, Adalimumab, Certolizumab pegol, Etanercept, Golimumab, and Infliximab and analogs and derivatives thereof. Exemplary IFN-γ inhibitors that can be administered according to the methods of the invention include, but are not limited to, anti-IFN-γ antibodies, glucocorticoids, Mesopram, GIT27, Rocaglamide, MAB2851, and analogs and derivatives thereof. In some embodiments, the combination of inhibitors inhibits the ability of leukocytes to migrate and interact with target cells. Exemplary leukocyte migration and functional inhibitors that can be administered according to the methods of the invention include, but are not limited to fingolimod, vendolizumab, and analogs and derivatives thereof.

In some embodiments, at least 2 compositions of the invention are administered concurrently. In some embodiments, at least 2 compositions of the invention are administered sequentially.

In one embodiment, a first inhibitor composition is administered one or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days, before a second inhibitor composition is administered. In one embodiment, the first inhibitor composition is administered one or more months, 2 or more months, 3 or more months, 4 or more months, 5 or more months, 6 or more months, 7 or more months, 8 or more months, 9 or more months, 10 or more months, 11 or more months, or 12 or more months, before the second inhibitor composition is administered.

In certain embodiments, the method comprises repeated administration of one or more of the compositions.

The administration of the subject compositions may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous (i.v.) injection, or intraperitoneally.

Forms of administration that may be useful in the methods described herein include, but are not limited to, direct delivery to a desired organ, oral, inhalation, intranasal, intratracheal, intravenous, intramuscular, intratumoral, subcutaneous, intradermal, and other parental routes of administration. Additionally, routes of administration may be combined, if desired. In one embodiments, route of administration is intradermal injection or intratumoral injection. In one embodiment, one or more composition is administered to a treatment site during a surgical procedure, for example during surgical resection of all or part of a tumor.

### Dosage and Formulation (Compositions)

Compositions of the present invention may be formulated and administered in a manner appropriate to the disease to be treated (or prevented). The quantity and frequency of administration will be determined by such factors as the condition of the patient, and the type and severity of the patient's disease, although appropriate dosages may be determined by clinical trials. When “an effective amount”, or “therapeutic amount” is indicated, the precise amount of the compositions of the present invention to be administered can be determined by a physician with consideration of individual differences in age, weight, disease progression, and condition of the patient (subject). The optimal dosage and treatment regime for a particular patient can readily be determined by one skilled in the art of medicine by monitoring the subject for signs of disease and adjusting the treatment accordingly.

The present invention envisions treating a disease, for example, diseases or disorders associated with immune response-mediated tissue injury, in a subject having an inactivating mutation in the Autophagy Related 16 Like 1 gene by the administration of one or more of the therapeutic agents of the present invention (e.g., a necroptosis or interferon signaling inhibitor, or a combination thereof).

In one embodiment, the present invention provides a method comprising administering one or more of the therapeutic agents of the present invention (e.g., a necroptosis or interferon signaling inhibitor, or a combination thereof) to a subject. In one embodiment, the method comprises administering one or more of the therapeutic agents of the present invention (e.g., a necroptosis or interferon signaling inhibitor, or a combination thereof) to a subject having a disease or disorder associated with immune response-mediated tissue injury. In one embodiment, the method comprises administering one or more of the therapeutic agents of the present invention (e.g., a necroptosis or interferon signaling inhibitor, or a combination thereof) to a subject having an inactivating mutation in the Autophagy Related 16 Like 1 gene. In one embodiment, the method comprises administering one or more of the therapeutic agents of the present invention (e.g., a necroptosis or interferon signaling inhibitor, or a combination thereof) to a subject, wherein the subject has a disease or disorder associated with immune response-mediated tissue injury and wherein the subject has an inactivating mutation in the Autophagy Related 16 Like 1 gene.

Administration of the composition in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. In one embodiment, the cytokine composition, the antigen receptor composition, and the integration composition of the invention are administered locally to the same site. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into a tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

In certain embodiments, the therapeutic agent is combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect. One skilled in the art will recognize that although more than one route can be used for administration, a particular route can provide a more immediate and more effective reaction than another route. In one embodiment, the composition described above is administered to the subject by intratumoral injection. Other forms of administration that may be useful in the methods described herein include, but are not limited to, direct delivery to a desired organ, intramuscular, subcutaneous, intradermal, and other parental routes of administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Gene Therapy Administration

One skilled in the art recognizes that different methods of delivery may be utilized to administer a nucleic acid molecule (e.g., a vector) into a cell. Examples include: (1) methods utilizing physical means, such as electroporation (electricity), a gene gun (physical force) or applying large volumes of a liquid (pressure); and (2) methods wherein the vector is complexed to another entity, such as a liposome, aggregated protein or transporter molecule.

Furthermore, the actual dose and schedule can vary depending on whether the compositions are administered in combination with other compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

The nucleic acid molecule may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

Organoids

In one embodiment, the invention relates to ex vivo culture platforms, and methods of using said ex vivo culture systems, wherein the ex vivo culture platform comprises organoids and/or organoid co-cultures obtained from epithelial cells of a subject. The ex vivo culture platforms allow for investigation of subject-specific responses of organoids and/or organoid co-cultures to various test agents or test conditions.

Organoids may be prepared by culturing normal epithelial cells in an organoid culture medium. In some embodiments, an organoid is a three-dimensional cellular structure. In some embodiments, an organoid is grown as a monolayer. In some embodiments, an organoid comprises a lumen surrounded by epithelial cells. In some embodiments, the epithelial cells surrounding the lumen are polarized. In some embodiments, the epithelial cells from which organoids are obtained are primary epithelial cells.

Organoids and/or organoid co-cultures may be obtained from normal (i.e. non disease) epithelial cells or from disease epithelial cells (sometimes specifically referred to as ‘disease organoids’ or ‘disease co-cultures’). Any epithelial cell from which an organoid can be generated is suitable for use in the invention. Epithelial cells include, but are not limited to, lung cells, liver cells, breast cells, skin cells, intestinal cells, crypt cells, rectal cells, pancreatic cells, endocrine cells, exocrine cells, ductal cells, renal cells, adrenal cells, thyroid cells, pituitary cells, parathyroid cells, prostate cells, stomach cells, esophageal cells, ovary cells, fallopian tube cells and vaginal cells. In one embodiment, the epithelial cells are intestinal cells, for example small intestinal and colonic crypt cells.

In one embodiment, the organoids are obtained from a subject from a specific population, such as a population characterized by its gender, weight, body-mass index, disease state, ethnicity, age, responsiveness to treatment, or genetics. For example, in certain embodiment, the subject from which the organoid is obtained has a specific genotype. In one embodiment, the subject has a genetic mutation. In one embodiment, the subject has an inactivation mutation in Atg16L1 (such as a T300A mutation in Atg16L1). However, the present ex vivo culture platform, and uses thereof, is not limited to any particular subject, but rather can be used to investigate subject-specific responses for any subject of interest.

In one embodiment, provided is a method for preparing an intestinal organoid culture or co-culture. In some embodiments, the method comprises culturing intestinal epithelial cells in contact with an extracellular matrix in an organoid culture medium comprising one or more additional agents to obtain an organoid culture or co-culture. In some embodiments, the method comprises culturing intestinal epithelial cells in contact with an extracellular matrix in an organoid culture medium, removing said extracellular matrix and organoid culture medium from said organoid, and resuspending said organoid in a cell culture medium supplemented with one or more additional agent.

The extracellular matrix used with the methods of preparing an intestinal organoid co-culture according to the invention may be a hydrogel, foam or non-woven fibre. In some embodiments, the matrix is a hydrogel.

In some embodiments, the organoid culture or co-culture medium comprises DMEM/F-12 supplemented with 100 IU penicillin, 100 μg/ml streptomycin, 125 μg/ml gentamicin, 2 mM 1-glutamine, 20 ng/ml mEGF, 100 ng/ml mNoggin, and 500 ng/ml mR-Spondin 1. In some embodiments, the organoid culture or co-culture medium further comprises 50 ng/ml Wnt-3a, 1×B-27, and N-2 supplement.

Exemplary additional agents that can be included in an organoid culture or co-culture of the invention include, but are not limited to, immune cells, and inflammatory cytokines, including, but not limited to interleukin-1 (IL-1), IL-12, IL-17, IL-22, and IL-18, tumor necrosis factor alpha (TNF-α), interferon gamma (IFNγ), IFNα, IFNβ and IFNλ and granulocyte-macrophage colony stimulating factor (GM-CSF).

In one embodiment, the ex vivo culture platform comprises a co-culture of organoids and at least one additional cell type. For example, in one embodiment, the co-culture comprises organoids and immune cells. In one embodiment, the co-culture is an intestinal organoid-immune cell co-culture. In one embodiment, the organoids and the at least one additional cell type are both from, or derived from, subjects of the same population. In one embodiment, the organoids and the at least one additional cell type are both from, or derived from, subjects of the same species. In one embodiment, the organoids and the at least one additional cell type are both from, or derived from, the same subject. In one embodiment, the co-culture comprises organoids from a subject and immune cells from the subject. In one embodiment, provided is a method for preparing an intestinal organoid-immune cell co-culture, wherein the method comprises: culturing intestinal epithelial cells in contact with an extracellular matrix in an organoid culture medium to obtain an organoid; removing said extracellular matrix and organoid culture medium from said organoid; re-suspending said organoid in immune cell culture medium supplemented with interleukin; preparing an immune cell suspension comprising immune cells, immune cell culture medium supplemented with interleukin, and collagen in the suspension; and mixing the immune cell suspension comprising immune cells with the resuspended organoid.

Immune Cells

Any immune cell that can be incorporated into a co-culture is suitable for use with methods of the invention. The immune cells can be leucocytes or co-cultures of leucocytes with other cells of interest. The leucocytes may be selected from the group consisting of whole peripheral blood mononuclear cells, defined subpopulations of peripheral blood mononuclear cells, in vitro differentiated peripheral blood mononuclear cell subpopulations and any co-cultures of these. Leucocytes comprise defined subpopulations of leucocytes; such as lymphocytes (T cells, B cells) monocytes and in vitro differentiated leucocytes and any co-cultures of these (e.g., T cell and dendritic cell co-culture or B/T cell and dendritic cell co-cultures). Furthermore, leucocytes or defined subpopulations of leucocytes may be co-cultivated with other cells of interest selected from the group consisting of endothelial cells, stem cells, follicular dendritic cells, stromal cells and others. In addition, cell lines with specific immunofunctions can be used. These cell lines are derived from immune cells and can mimic immune responses. These cells can be selected from a group consisting of B cell lines (e.g. Ramos, Raji), T cell lines (e.g. Jurkat, Karpas-299) or dendritic cell lines (e.g. Nemod), or others known to the skilled person. In some embodiments, mixtures of these cell lines can be cultured.

The immune cells may be obtained from established cell lines available in the art (e.g. from ATCC or similar libraries of cell lines). Alternatively, the immune cells may be purified from an impure sample from a subject. An impure immune sample from which immune cells may be obtained, may include intestinal tissue and/or peripheral blood. In some embodiments, the immune cells are obtained from a peripheral blood sample and/or a tissue biopsy. For example, peripheral blood lymphocytes (PBLs) and/or T cells may be obtained from a peripheral blood sample; or tumor-infiltrating lymphocytes (TILs) and/or intra-epithelial lymphocytes (IELs) are obtained from a healthy tissue biopsy.

Immune cells suitable for use in methods of the invention may be allogeneic with the organoid. In some embodiments, the immune cells are HLA-matched with the organoid, that is, the immune cells may be antigenically compatible with the patient from whom the organoid is derived. In some embodiments, the immune cells in the co-culture are engineered T cells, such as CAR-T cells.

The intestinal organoid immune cell co-culture of the invention and corresponding methods allows emulating immunogenicity and immune functions in vitro. In some embodiments, the intestinal organoid immune cell co-culture system of the invention allows mimicking immunological functions and testing immunogenicity in vitro and is aimed for testing the effects of substances as drugs and immunological stimulators on immune cells and co-cultures of immune cells and intestinal cells.

Methods of Evaluating Responsiveness or Susceptibility

In one embodiment, the invention provides methods of evaluating the responsiveness of an intestinal organoid culture or co-culture to an agent of interest. For example, in one embodiment, the method comprises contacting an intestinal organoid culture or co-culture with an agent of interest and detecting one or more change in the organoid culture or co-culture indicative of a response to the agent. For example, in some embodiments, the detected change can be based on identification of an increase or decrease in cell viability, organoid size (e.g., surface area), morphology (e.g., budding), an alteration in protein levels or post-translational modifications of proteins, metabolism, production of soluble factors, an alteration in the quantification of epithelial subsets, including but not limited to, Paneth cells, goblet cells, stem cells, tuft cells, neuroendocrine cells, and enterocytes, cell proliferation, or any combination thereof, of the intestinal organoid cells as compared to a comparator control.

In one embodiment, the method comprises evaluating the susceptibility of the intestinal organoid culture or co-culture to injury mediated by an agent. For example, in one embodiment, the method comprises contacting an intestinal organoid culture or co-culture with an agent of interest and detecting a change in cell health or cell viability. For example, in some embodiments, the detected change can be based on identification of an increase or decrease in cell viability, organoid size (e.g., surface area), morphology (e.g., budding), an alteration in protein levels or post-translational modifications of proteins, metabolism, production of soluble factors, an alteration in the quantification of epithelial subsets, including but not limited to, Paneth cells, goblet cells, stem cells, tuft cells, neuroendocrine cells, and enterocytes, cell proliferation, or any combination thereof, of the intestinal organoid cells as compared to a comparator control. In one embodiment, the method is used to predict whether the subject from which the intestinal organoid is derived would be responsive to a treatment that targets or inhibits the agent of interest. For example, in one embodiment, the method comprises deriving or obtaining an intestinal organoid of a subject having or suspected of having a disease or disorder associated with immune-mediated tissue injury; contacting the organoid culture or co-culture with an agent of interest; detecting that the agent causes decreased cell viability or other marker indicative of cell injury or decreased cell health; and thereby identifying that the subject would be responsive to a treatment that targets or inhibits the test agent. In one embodiment, the subject has or is suspected of having a disease or disorder associated with immune-mediated tissue injury, including but not limited to intestinal graft-versus-host disease (GVHD), inflammatory bowel disease (IBD), Crohn's disease (CD), ulcerative colitis (UC), pouchitis, irritable bowel syndrome (IBS), infectious and non-infectious gastroenteritis, autoimmunity associated with cancer immunotherapy, gastrointestinal cancer (e.g., esophageal, colorectal, small intestine, oral, and gastric cancer), or radiation enteritis. In one embodiment, the method comprises administering the treatment that inhibits or targets the agent to the subject when it is detected that the organoid of the subject is susceptible to the agent.

For example, in one embodiment, the present invention provides a method of predicting or evaluating a subject's responsiveness to anti-TNFα treatment, comprising obtaining or deriving an intestinal organoid from the subject, contacting the intestinal organoid culture or co-culture with TNFα, and detecting decreased cell viability in response to TNFα; thereby indicating that the subject would be responsive to anti-TNFα treatment. In one embodiment, the subject has or is suspected of having a disease or disorder associated with immune-mediated tissue injury, including but not limited to intestinal graft-versus-host disease (GVHD), inflammatory bowel disease (IBD), Crohn's disease (CD), ulcerative colitis (UC), pouchitis, irritable bowel syndrome (IBS), infectious and non-infectious gastroenteritis, autoimmunity associated with cancer immunotherapy, gastrointestinal cancer (e.g., esophageal, colorectal, small intestine, oral, and gastric cancer), or radiation enteritis. In one embodiment, the method comprises administering an anti-TNFα treatment to the subject when it is detected that the organoid of the subject is susceptible to TNFα.

In certain embodiments, the intestinal organoids of the culture or co-culture are from, or derived from, a subject harboring an inactivating mutation in ATG16L1.

Methods of Testing Therapeutic Agents

In one embodiment, the invention provides methods of analyzing the effect of a test compound on a specific tissue microenvironment (e.g., an intestinal microenvironment). In some embodiments, the invention provides methods for identifying a therapeutic agent for the treatment of a disease or disorder associated with immune response-mediated tissue injury, comprising contacting an intestinal organoid culture or co-culture with one or more candidate agents and detecting the presence or absence of one or more change in the intestinal organoid culture or co-culture that is indicative of therapeutic efficacy. In some embodiments, a candidate agent is identified as a therapeutic agent based on identification of an increase in cell viability, organoid size (e.g., surface area), morphology (e.g., budding), an alteration in protein levels or post-translational modifications of proteins, metabolism, production of soluble factors, an alteration in the quantification of epithelial subsets, including but not limited to, Paneth cells, goblet cells, stem cells, tuft cells, neuroendocrine cells, and enterocytes, cell proliferation, or any combination thereof, of the intestinal organoid cells as compared to a comparator control. In some embodiments, a candidate agent is identified as a therapeutic agent if there is at least one change in the transcriptome, proteome or secretome of the intestinal organoid cells as compared to a comparator control. In certain embodiments, the intestinal organoids of the culture or co-culture are from, or derived from, a subject harboring an inactivating mutation in ATG16L1.

### Kits

The invention also includes a kit comprising one or more of the compositions described herein. For example, in one embodiment, the kit comprises one or more of: a necroptosis or interferon signaling inhibitor as described herein. In one embodiment, the kit comprises an organoid co-culture as described herein. In one embodiment, the kit comprises instructional material which describes the use of the composition. For instance, in some embodiments, the instructional material describes administering the composition(s), to a subject as a therapeutic treatment or a non-treatment use as described elsewhere herein. In some embodiments, this kit further comprises a (optionally sterile) pharmaceutically acceptable carrier suitable for dissolving or suspending the composition(s), for instance, prior to administering the composition(s) to a subject. Optionally, the kit comprises an applicator for administering the composition(s).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: An Intestinal Organoid-Based Platform that Recreates Susceptibility to T Cell-Mediated Tissue Injury

Although many autoimmune and inflammatory disorders are driven by lymphocytes and their effector molecules, it remains possible that differential susceptibility of target tissues to injury underlies heterogeneity in patients. Identifying the pathways and genetic factors that contribute to the resilience of the parenchyma to immune-mediated destruction has the potential to aid prognosis and facilitate individualized therapy. The experiments presented herein demonstrate that ATG16L1 has a conserved function in protecting IECs from killing by allogeneic T cells, such as those encountered following allo-HCT. The experiments also demonstrate that ATG16L1 inhibits intestinal GVHD and promotes survival in an improved preclinical allo-HCT model by preventing necroptosis of IECs. Excess cell death in the intestinal epithelium due to dysregulated RIP1 and RIP3 signaling has been linked to intestinal inflammation (Takahashi et al., 2014, Nature 513, 95-99; Cuchet-Lourenco et al., 2018, Science 361, 810-813; Dannappel et al., 2014, Nature 513, 90-94), raising the possibility that targeting these molecules may be effective for treatment of GVHD or IBD. Inducing necroptosis by deleting caspase-8 in IECs is sufficient to induce a lethal inflammatory disease in mice along with Paneth cell depletion, and hallmarks of necroptosis have been observed in Crohn's disease patients (Gunther et al., 2013, Gut 62, 1062-1071; Stolzer et al., 2019, Inflamm Bowel Dis. izz142). Accordingly, it was found that genetic and chemical inhibition of RIP3 and RIP1, respectively, ameliorated GVHD in allo-HCT recipient Atg16L1^{ΔIEC} mice. Also, decreased Paneth cell numbers, a hallmark of intestinal GVHD in humans (Levine et al., 2013, Blood 122, 1505-1509), were reversed in Atg16L1^{ΔIEC} mice upon inhibition of necroptosis. Therefore, targeting the RIP1/3 signaling complex in patients with intestinal GVHD may be an effective means to ameliorate symptoms.

Based on the in vivo results in the animal model, an ex vivo GVHD platform was deigned that reproduced the role of ATG16L1 in IEC survival remarkably well. Organoids derived from the small intestine of Atg16L1^{ΔIEC} mice and ATG16L1^{T300A} homozygous humans were susceptible to necroptosis induced by allogeneic T cells in this co-culture model that was developed. Notably, initially the experiments were performed with human organoids blind to genotype rather than selecting for ATG16L1^{T300A} homozygous samples. In the example of this current study, mechanistic experiments in the mouse model led to the hypothesis that the susceptibility was driven by a particular gene variant, and further helped predict drug targets. These findings provide proof-of-principle for a general approach in which heterogeneous responses to T cell-mediated injury can be recreated in a quantitative ex vivo assay that can be subsequently used to identify variables that contribute to this inter-individual variation.

In addition to establishing a pipeline, the results may have specific implications for the treatment of intestinal GVHD. It is unclear whether TNFα blockade is efficacious for treating intestinal GVHD, and the role of IFNγ in GVHD is complex (Wang and Yang, 2014, Immunol Rev, 258:30-44). When allogeneic T cells were co-cultured with Atg16L1^{ΔIEC} organoids, the combination of anti-TNFα and anti-IFNγ antibodies were required for full restoration of viability. Excess IL-22 has also been shown to induce necroptosis in ATG16L1-deficient IECs (Aden et al., 2018, J Exp Med, 215:2868-2886). TNFα, IFNγ, and IL-22 were all increased in the culture supernatant, albeit the increase in IL-22 was only observed when the organoids were specifically co-cultured with B10.BR T cells and not LP/J T cells. It is possible that in intestinal GVHD patients, these and potentially other cytokines have redundant functions, and that blocking any individual cytokine would be insufficient to ameliorate disease. In this scenario, enhancing the resilience of the intestinal barrier to damage would be more efficacious.

Another important observation in this study is that T cells can kill target cells through necroptosis. This key finding raises two important future research directions. First, it is unclear whether T cell-induced necroptosis is specific to pathophysiological conditions (alloreactivity), or whether this process can benefit the host, such as through elimination of virally-infected cells or tumors. Second, necroptosis rarely occurs in vitro under physiological conditions; cell culture models of necroptosis typically require shunting of the pathway downstream of TNFα away from apoptosis, such as through inhibition of caspase-8. Thus, how ATG16L1 mutation overcomes this barrier for necroptosis is of great interest. The results presented in this manuscript, together with recent literature, offer some insight. Inhibition of ATG16L1 leads to spontaneous STAT1 activation and ISG expression in IECs in vivo and in vitro in a manner dependent on mitochondria-associated antiviral signaling (MAVS) and stimulator of interferon genes (STING), two signaling adaptors involved in sensing of viral nucleic acid (Aden et al., 2018, J Exp Med, 215:2868-2886; Martin et al., 2018, Nat Microbiol 3, 1131-1141). ATG16L1 and autophagy also has a role in targeting the degradation of TRIF, an adaptor molecule involved in viral recognition (Samie et al., 2018, Nat Immunol, 19:246-254), and Z-DNA-binding protein 1 (ZBP1, also known as DAI or DLM-1) interacts with RIP3 to sensitize cells to virus-induced necroptosis (Kuriakose et al., 2016, Sci Immunol 1, aag2045; Lim et al., 2019, Elife 8:e44452; Upton et al., 2012, Cell Host Microbe, 11:290-297). More recently, IFN-λ, which also signals through STAT1, was shown to exacerbate necroptosis in intestinal epithelial cells (Gunther et al., 2019, Gastroenterology, S0016-5085(19)41128-1). Therefore, it is possible that inhibiting autophagy in IECs sensitizes cells to necroptosis by mimicking aspects of viral infection, such as activation of interferons and JAK/STAT signaling.

Interestingly, the results suggest that disrupting these pathways predominantly affects the small intestinal epithelium rather than the colon. Allogeneic T cells have been observed to preferentially migrate to the small intestinal crypt to destroy Paneth cells and the stem cell niche (Hanash et al., 2012, Immunity 37:339-350), and a similar depletion of Paneth cells was observed in the Atg16L1 mutant setting. Traditionally, the diagnosis and evaluation of intestinal GVHD has relied on examination of the upper gastrointestinal tract or distal colon (Thompson et al., 2006, Bone Marrow Transplant, 38:371-376; Velasco-Guardado et al., 2012, Rev Esp Enferm Dig, 104:310-314). However, recent studies have highlighted the utility of sampling the small intestine when diagnosing or predicting the severity of intestinal GVHD (Ip et al., 2016, Dig Dis Sci, 61:2351-2356; Sugihara et al., 2018, BMC Gastroenterol, 18:111). Given that recent technical advances in balloon or capsule endoscopy has enabled easy access to the small intestine, it may be prudent to analyze the small intestine in allo-HCT recipients.

Finally, the results raise the possibility that ATG16L1^{T300A} homozygous individuals are more likely to respond to therapies targeting RIP 1 or JAK/STAT signaling, both of which are in clinical trials for several diseases. Repurposing these drugs for treating intestinal GVHD or Crohn's disease may be worth considering, especially if they can be targeted to likely responders. In conclusion, the data suggest that advanced cell culture techniques that involve growing parenchymal cells together with lymphocytes or their effector molecules can recreate inter-individual heterogeneity to tissue injury, which is a hallmark of a variety of disorders. This approach can be applied to multiple tissues. Both parenchymal and lymphocyte specimens can be derived directly from the patient cohort of interest to predict a given individual's susceptibility to injury for the purpose of prognosis or drug responsiveness.

The materials and methods used in the experiments are now described

Study Design

This study was designed with two objectives: (1) to examine the role of necroptosis signaling in intestinal GVHD in the presence of Atg1611 mutation, and (2) to develop an ex vivo model that will complement animal studies to investigate mechanisms that mediate susceptibility to intestinal GVHD. An improved preclinical animal model was used that incorporated Atg16L1 mutant mouse lines and littermate controls to demonstrate a role for necroptosis in the exacerbated disease that occurs upon inhibition of this gene. Next, an ex vivo assay was established in which intestinal organoids are co-cultured with lymphocytes and assayed for viability. RNA Sequencing analysis was performed to gain mechanistic insight, and tested the role of a putative signaling pathway with a chemical inhibitor. Based on results derived from the animal model and cell derived from mice, an analogous ex vivo co-culture model of GVHD was developed using human material. The sample size per experiments is included in the figure legends. The number of mice used was selected based on previous studies using animal GVHD model and calculating the statistical power, considering a minimum of six mice in total pooled from at least two independent experiments. In animal allo-HCT experiments, mice that reached a clinical score of 8 were ethically euthanized. All animal experiments were performed according to approved protocols. In experiments using human material, endoscopic biopsy specimens and peripheral blood mononuclear cells (PBMCs) were collected. The endoscopic biopsy specimens and their corresponding blood samples were acquired from random adult IBD patients who underwent endoscopy during routine visits to the hospital with signed consent. PBMCs from 20 healthy volunteers were harvested separately for allogeneic T cells. In allo-HCT, the animal caretakers and investigators conducting the experiments were blinded to the genotyping and condition of the mice. Quantification of all microscopy data was performed blind. The genotyping of human samples was performed retrospectively after the ex vivo analyses were completed.

Mice

Age- and gender-matched 6-15 weeks old mice on the C57BL/6J (B6) background were used as recipients. Atg16L1^{f/f}; villinCre (Atg16L1^{ΔIEC}) and littermate control Atg16L1^{f/f} mice were generated as previously described (9). Atg16L1^{f/f}×Rip3^{-/-} (f/f Rip3^{-/-}) and Atg16L1^{ΔIEC}×Rip3 (ΔIEC Rip3^{-/-}) mice were generated for experiments by crossing Atg16L1^{ΔEIC} mouse with RIP3^{-/-} mouse provided by Xiaodong Wang (National Institute of Biological Sciences). B6, B10.BR, and LP/J mice were purchased from The Jackson Laboratory and bred onsite to generate animals for experimentation. Atg4B^{-/-} mice were provided by Skip Virgin (Washington University School of Medicine). Atg16L1^{T316A} mice were provided by M. van Lookeren Campaigne (Genentech). All animal studies were performed according to approved protocols.

Endoscopic Specimens

Pinch biopsies were obtained from adult IBD patients undergoing surveillance colonoscopy using 2.8 mm standard biopsy forceps (Mucosal Immune Profiling in Patients with Inflammatory Bowel Disease; S12-01137). All biopsies were collected in ice cold complete RPMI (RPMI 1640 medium suppled with 10% fetal bovine serum (FBS), penicillin/streptomycin/glutamine, and 50 μM 2-mercaptoethanol). Inflammation status of tissue included in the study was confirmed by pathological examination.

Preparation of PBMCs

To harvest allogeneic T cells, peripheral blood mononuclear cells (PBMCs) from anonymous, healthy donors (New York Blood Center) were isolated by Ficoll gradient separation as previously described (Reyes-Robles et al., 2016, EMBO Rep 17, 780). CD14+ monocytes were then removed from the PBMC fraction by positive selection. The remaining negative fraction was used to isolate T cells.

To harvest syngeneic T cells, venous blood was collected at the time of endoscopic procedures n sodium heparin BD Vacutainer blood collection tubes (Becton Dickinson).

Hematopoietic Cell Transplantation

Allogeneic hematopoietic cell transplantation (allo-HCT) was performed according to the previous study (Riesner et al., et al., 2016, Bone Marrow Transplant 51, 410-417). In brief, female B6 background mice were intraperitoneally injected with busulfan (20 mg/kg/day) for 5 days, followed by cyclophosphamide (100 mg/kg/day) for 3 days. Days-2 and -1 were rest days. The recipient mice were intravenously injected 5×10⁶ bone marrow (BM) cells after T cell depletion with anti-Thy-1.2 and low-TOX-M rabbit complement (Cedarlane Laboratories). Donor T cells were prepared by harvesting splenocytes and enriching T cells by Miltenyi MACS purification of CD5 (routinely >90% purity). Recipient mice were monitored daily for survival and weekly quantified for clinical GVHD for 5 clinical parameters (weight loss, hunched posture, activity, fur ruffling, and skin lesions) on a scale from 0 to 2. A clinical GVHD score was generated by the summation of the 5 criteria as previously described (Cooke et al., 1996, Blood 88, 3230-3239).

Histology and Immunohistochemistry

Intestinal sections were prepared and stained with H&E and PAS/Alcian blue as previously described (Matsuzawa-Ishimoto et al., 2017, J Exp Med 214, 3687-3705). Histopathology was scored by C. L. as previously described (Hubbard-Lucey et al., 2014, Immunity 41, 579-591; Matsuzawa-Ishimoto et al., 2017, J Exp Med 214, 3687-3705). Immunohistochemistry (IHC) for Cleaved Caspase-3 and TUNEL staining was performed as previously described (Matsuzawa-Ishimoto et al., 2017, J Exp Med 214, 3687-3705). Appropriate positive and negative controls were run in parallel to study sections. At least 50 crypt-villus axes per mouse were observed to count Paneth cells, goblet cells, cleaved caspase-3⁺ and TUNEL⁺ cells. Histology and IHC samples were analyzed using Zeiss Axioplan with Spot camera. Microscopic analyses of live organoids were performed using a Zeiss AxioObserver.Z1 with Zen Blue software (Zeiss) and EVOS FL Auto (Life Technologies). Images were processed and quantified using ImageJ.

Flow Cytometry and Cytokine Analyses

Anti-mouse antibodies were obtained from BioLegend (CD4, CD8a, B220, CD11b, I-AE, Ly-6G/Ly-6C (Gr-1), CD25, and H-2Ld/H-2db (MHC class I)), eBioscience (CD11c), and Invitrogen (Foxp3 and DAPI). Anti-human antibodies were obtained from BioLegend (CD3, CD4, and CD8α). Cells were stained for 20 min at 4° C. in PBS with 0.5% bovine serum albumin (BSA) (PBS/BSA) after Fc block (Bio X CELL), washed, fixed with Fixation Buffer (Biolegend) or Foxp3/Transcription Factor Staining Buffer Set (Invitrogen) according to the manufacturer's protocol, and resuspended in PBS/BSA. To exclude dead cells, Zombie UV Fixable Viability Kit (BioLegend) or DAPI (Invitrogen) were used. Flow cytometry was performed on an LSR II (BD Biosciences) and analyzed with FlowJo (Tree Star Software). For cytokine quantification, blood was collected into microcentrifuge tubes, allowed to clot, and centrifuged, and the serum was collected. The culture supernatant was harvested at 48 hours after co-culture. ProcartaPlex Multiplex Immunoassay was conducted per the manufacturer's instructions (Affymetrix). Results were acquired with a Luminex 200 instrument and analyzed with xPONENT software (Luminex Corporation).

Bacterial Translocation Assay

Spleens were weighed and homogenized in sterile PBS. Serial dilutions of the homogenates were plated on blood agar plates and colonies were quantified following up to 24 hours incubation at 37° C. Bacterial titers are shown as cfu/g.

Genotyping of Human Tissues

Genomic DNA was extracted either from whole venous blood using the QIAamp Mini Blood Kit (QIAGEN) or grown intestinal organoids using NucleospinSoil Kit (Macherey-Nagel) according to the manufacturer's protocol. Genotyping of each DNA was performed using Infinium Global Screening Array-24 Kit (Illumina).

Isolation of Murine and Human Cells

Murine CD5⁺ splenic lymphocytes were isolated from splenocytes with MACS CD5 (Ly-1) MicroBeads (Miltenyi Biotec) according to the manufacturer's protocol. Human naïve T cells were isolated from PBMCs with Dynabeads™ Untouched™ Human T cells (Invitrogen) according to the manufacturer's protocol. Sorted human T cells were frozen with FBS containing 10% dimethyl sulfoxide (Fisher Scientific) in liquid nitrogen tank until the day of co-culture experiment.

Intestinal Organoids

Mouse small intestinal and colonic crypts were isolated and cultured. Crypts of proximal small intestine were counted and embedded in Matrigel at 10,000 crypts/ml and cultured in DMEM/F-12 in the presence of 100 IU penicillin and 100 μg/ml streptomycin (Corning), 125 μg/ml gentamicin (Gibco), 2 mM 1-glutamine (Corning), 20 ng/ml mEGF (PeproTech), 100 ng/ml mNoggin (R&D Systems), and 500 ng/ml mR-Spondin 1 (R&D Systems), hereafter referred as ENR medium. Crypts of colon were counted and embedded in Matrigel and cultured in the ENR medium plus 50 ng/ml Wnt-3a, 1×B-27, and N-2 supplement (Thermo Fisher Scientific). Surface area of organoids was quantified with Image J. For organoid viability assays, crypts were embedded in 10 IA of Matrigel at 5,000 crypts/ml and cultured in 96-well culture plate in triplicate with or without 20 ng/ml mTNFα (PeproTech) and 100 nM Ruxolitinib (Selleckchem). Percent viable organoids was determined by daily quantification of the number of intact organoids. Opaque organoids with condensed structures or those that have lost adherence were excluded. Dead organoids were marked by staining with 100 μg/ml propidium iodide (SIGMA-ALDRICH). Human intestinal organoids were cultured (Neil et al., 2019, Nat Microbiol 4, 1737-1749). Grown human organoids were frozen with FBS containing 7% dimethyl sulfoxide (Fisher Scientific) in liquid nitrogen tank until the day of co-culture experiment. Thawed organoids were cultured until they turned out to proliferate well. For human organoid viability assay, mature organoids were embedded in 10 IA of Matrigel at 5,000/ml and cultured in 96-well culture plate in triplicate with or without 50 ng/ml human TNFα (PeproTech), 100 nM Ruxolitinib, 1 μM GSK547, 2 μM Necrostatin-1s (Nec-1s) (BioVision), and 2 μM Necrosulfonamide (NSA) (Millipore Sigma) (Sun et al., 2012, Cell 148, 213-227).

Ex Vivo GVHD Model

Mouse organoids at day 3 or mature human organoids were released from Matrigel (Corning) using Cell Recovery Solution (Corning) and incubated on ice for 45 min. Both murine and human T cells were stimulated with 1× Cell Stimulation Cocktail (eBioscience) for 2 hours at 37° C. before co-culture. About 150-300 organoids and either 5×10⁵ mouse or 2.5×10⁵ human T cells were intermixed in 1.5 mL tube with 1 mL of DMEM. After incubation at 37° C. for 5 min, the mixture was centrifuged either for 2 min at 200 g or for 3 min at 300 g in mouse or human organoids, respectively. The pellet was suspended in 50 lit of Matrigel, and each 10 lit drop was placed in 96-well plates. After Matrigel polymerization, 100 μL of culture medium suppled with 10% FBS was added to each well. In some experiments, T cells were stained with CellBrite™ Green (Biotium) according to the manufacturer's protocol before co-cultured with organoids.

Immunoblotting

In mouse samples, isolated small intestinal crypts were cultured in ENR medium±100 nM Ruxolitinib for 3 days. Organoids were stimulated with 20 ng/ml mTNFα for 2 hours, released from Matrigel and incubated in lysis buffer (20 mM Tris-HCl (pH 7.4), 150 mM NaCl, 1% Triton X-100, 10% glycerol, and 1× Halt protease and phosphatase inhibitor cocktail (ThermoFisher Scientific)) on ice for 20 min, and centrifuged at 15,000 g for 20 min. Samples were resolved on Bolt 4-12% Bis-Tris Plus Gels (Invitrogen) and transferred to PVDF membranes. The following antibodies were used for immunoblotting studies: anti-β-actin (AC-15, Sigma-Aldrich), anti-RIP3 (phospho 5232) (Abcam), anti-RIP3 (AHP1797, AbD Serotec), anti-RIP (D94C12, Cell Signaling Technology), anti-Atg16L (M150-3, MBL). Secondary antibodies (mouse anti-rabbit and goat anti-mouse, 211-032-171 and 115-035-174, respectively) were purchased from Jackson Laboratories.

Quantitative RT-PCR

Total RNA was extracted from organoids treated with ±100 nM Ruxolitinib for 3 days using RNeasy Mini Kit (QIAGEN), and cDNA was synthesized using ProtoScript First Strand cDNA Synthesis kit (NEB) according to the manufacturer's protocol. Quantitative PCR was performed on a Roche 480 II LightCycler. Gene expression was normalized to Actb using the following primers; Oas12, 5′-GGATGCCTGGGAGAGAATCG-3′ (SEQ ID NO:1) and 5′-TCGCCTGCTCTTCGAAACTG-3′ (SEQ ID NO:2); Isg15, 5′-GGTGTCCGTGACTAACTCCAT-3′ (SEQ ID NO:3) and 5′-TGGAAAGGGTAAGACCGTCCT-3′ (SEQ ID NO:4); Apo19-a, 5′-GTGGATAGGATTGCCAGCAAG-3′ (SEQ ID NO:5) and 5′-AGAGGGGTTCCTTTCAGACTG-3′ (SEQ ID NO:6); Actb, 5′-CGGTTCCGATGCCCTGAGGCTCTT-3′ (SEQ ID NO:7) and 5′-CGTCACACTTCATGATGGAATTGA-3′ (SEQ ID NO:8).

RNA Sequencing and Analysis

Small intestinal organoids from Atg16L1^{f/f} and Atg16L1^{ΔIEC} mice were cultured for 3 days, and were treated ±20 ng/ml TNFα for 2 hours before RNA extraction. Organoids were released from Matrigel using Cell Recovery Solution (Corning), and total RNA was extracted using TRIzol (ThermoFisher) according to the manufacturer's protocol. RNA was prepared using RiboMinus (Life Technologies). Aligned RNA was analyzed for fold change. Sequencing data were processed using the Tuxedo suite; reads were aligned with TopHat v2.1.1 against UCSC mm10 genome assembly and normalized read counts were calculated using Cufflinks v2.2.1 against the same reference genome. Differential gene expression was determined with Cuffdiff and gene ontology analysis performed using Qiagen's Ingenuity Pathway Analysis (IPA, QIAGEN).

Statistical Analysis

GraphPad Prism version 7 was used for statistical analysis. Differences between two groups were assessed by two-tailed unpaired t test when data was distributed normally. An ANOVA with Tukey's multiple comparisons test was used to evaluate experiments involving multiple groups. Survival was analyzed with the Mantel-Cox log rank test. Continuous variables were analyzed by student's T-test and categorical variables were analyzed by chi-square or Fisher's exact test.

The results of the experiments are now described.

ATG16L1 in IECs Protects Against Lethal GVHD Mediated by RIP1 and RIP3

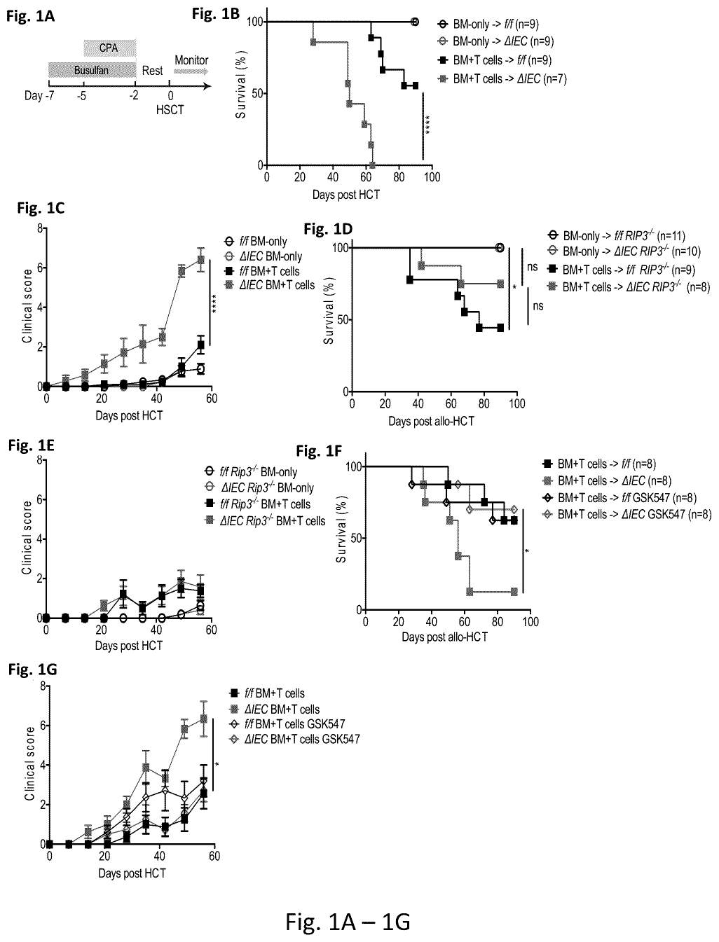

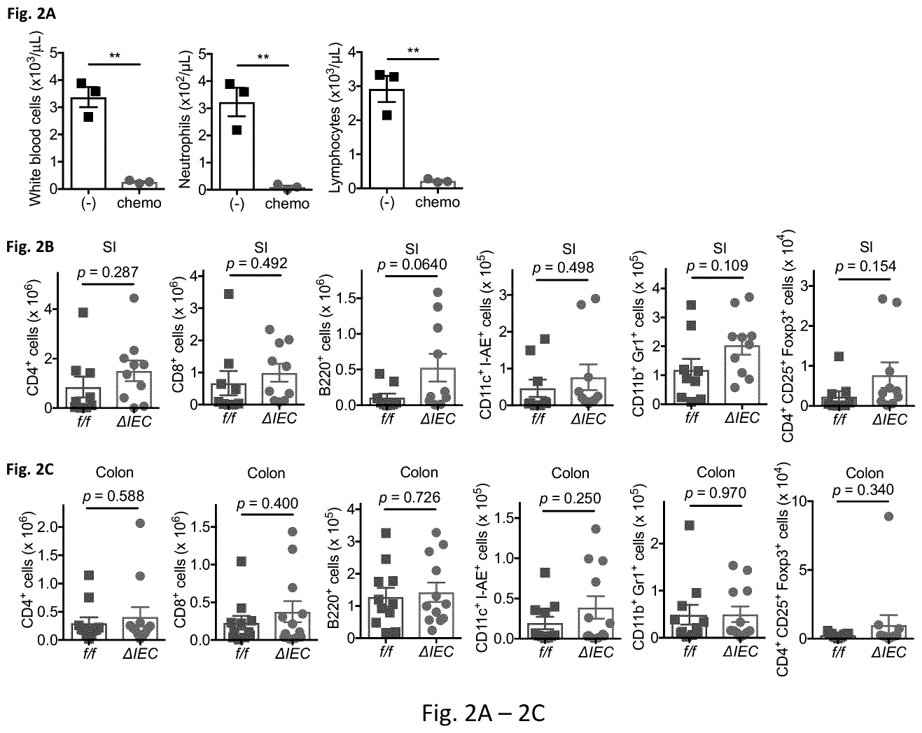

In a previous study, mice were generated with an IEC-specific deletion of Atg16L1 on the C57BL6/J (B6) background (H-2^{b}) by crossing villin-Cre and Atg16L1^{f/f} mice (Atg16L1^{ΔIEC}) and found that they displayed poor survival in an allo-HCT model where recipients are irradiated and injected with BM and T cells from donor B10.BR mice (H-2^{k}) (Matsuzawa-Ishimoto et al., 2017, J Exp Med 214, 3687-3705). Although this WIC-disparate model of allo-HCT was useful in identifying an IEC-intrinsic function of ATG16L1, the rapid onset of lethality that was observed suggests that this transplant procedure may not accurately reflect the course of GVHD in humans. Therefore, whether the protective function of ATG16L1 can be detected in an improved preclinical model that more closely recreates MHC-matched allo-HCT in cancer patients (Riesner et al., 2016, Bone Marrow Transplant 51, 410-417) was investigated. In this model, recipient mice were treated with busulfan and cyclophosphamide to mimic a chemotherapy-based conditioning regimen, which depletes leukocytes (P<0.01), and then injected with BM and T cells derived from LP/J mice (H-2 b) (FIG. 1A and FIG. 2A). Similar to previous findings, Atg16L1^{ΔIEC} recipient mice displayed 100% mortality and an increased clinical GVHD score (P<0.0001) compared with the Cre-negative Atg16L1^{f/f} control littermates, whereas all mice of both genotypes that received BM without T cells survived (FIG. 1B and FIG. 1C). This shows that Atg16L1 expression in IECs inhibits GVHD following allo-HCT.

Next, immune profiling was performed by quantifying the intestinal immune cell populations by flow cytometry and serum cytokines on day 28 after allo-HCT, a time point directly before the onset of lethality. A significant effect of ATG16L1 deficiency on the amount of specific immune cells or cytokines was not detected, with the exception of a <2-fold increase in IP-10 (CXCL10) (P<0.05) (FIG. 2B and FIG. 2C, and Table 1). These results suggest that, rather than skewing the immune response, deletion of ATG16L1 compromises the ability of IECs to withstand damage. To test whether worsened disease is dependent on necroptosis signaling, RIP3-deficient Atg16L1^{ΔIEC} mice (Atg16L1^{ΔIEC} Rip3^{-/-}) were generated and Cre-negative control mice (Atg16L1^{f/f} Rip3^{-/-}) were generated for comparison. Most Atg16L1^{ΔIEC} Rip3^{-/-} mice survived allo-HCT and displayed a similar degree of disease as Atg16L1^{f/f} Rip3^{-/-} mice (FIG. 1D and FIG. 1E). Additionally, Atg16L1^{ΔIEC} mice treated with the RIP1 inhibitor GSK547 displayed significantly better survival and clinical score (P<0.05) that was comparable to Atg16L1^{f/f} Rip3^{-/-} mice (FIG. 1F and FIG. 1G). These data indicate that ATG16L1 protects against lethal GVHD by preventing RIP1 and RIP3-mediated necroptosis of IECs.

TABLE 1
Quantification of Cytokines in Serum from Mice Deficient in
ATG16L1 in the Epithelium after Allo-HCT

| Serum | f/f (n = 11) | ΔIEC (n = 12) | |
|---|---|---|---|
| cytokine | Mean ± SEM (pg/ml) | | P-value |
| IL-10 | 10.93 ± 3.715 | 16.49 ± 2.777 | n.s. |
| IL-1β | 5.397 ± 1.201 | 4.511 ± 0.6468 | n.s. |
| IL-2 | 10.78 ± 5.18 | 13.29 ± 4.54 | n.s. |
| IP-10 | 43.94 ± 10.01 | 79.99 ± 13.32 | * |
| IL-4 | 2.463 ± 0.8634 | 5.319 ± 3.589 | n.s. |
| IL-5 | 24.65 ± 3.211 | 26.42 ± 3.525 | n.s. |
| IL-6 | 17.38 ± 3.225 | 18.86 ± 3.631 | n.s. |
| IL-22 | 101.7 ± 28.28 | 86.68 ± 27.97 | n.s. |
| IL-9 | 82.7 ± 18.98 | 66.46 ± 11.64 | n.s. |
| IL-13 | 9.857 ± 1.464 | 15.4 ± 3.467 | n.s. |
| IL-27 | 9.712 ± 1.544 | 15.94 ± 7.391 | n.s. |
| IL-23 | 31.31 ± 18.78 | 29.46 ± 9.991 | n.s. |
| IFNγ | 2.375 ± 0.4116 | 3.957 ± 1.079 | n.s. |
| GRO-a | 20.01 ± 4.315 | 22.67 ± 1.361 | n.s. |
| RANTES | 30.09 ± 6.494 | 46.2 ± 10.28 | n.s. |
| TNFα | 19.57 ± 6.081 | 24.25 ± 15.51 | n.s. |
| MIP-1a | 3.133 ± 0.473 | 4.306 ± 0.81 | n.s. |
| MCP-3 | 111.2 ± 35.87 | 87.12 ± 18.34 | n.s. |
| MCP-1 | 67.22 ± 9.368 | 63.95 ± 7.589 | n.s. |
| IL-17A | 11.45 ± 2.9 | 7.859 ± 2.079 | n.s. |
| MIP-2 | 26.95 ± 3.523 | 35.33 ± 10.96 | n.s. |
| Eotaxin | 699.5 ± 139.8 | 870.6 ± 112.7 | n.s. |
| IL-18 | 738.2 ± 152 | 911.7 ± 123.2 | n.s. |
| MIP-1b | 4.457 ± 1.09 | 3.766 ± 0.7553 | n.s. |

ATG16L1 Prevents Intestinal GVHD by Inhibiting Necroptosis of IECs

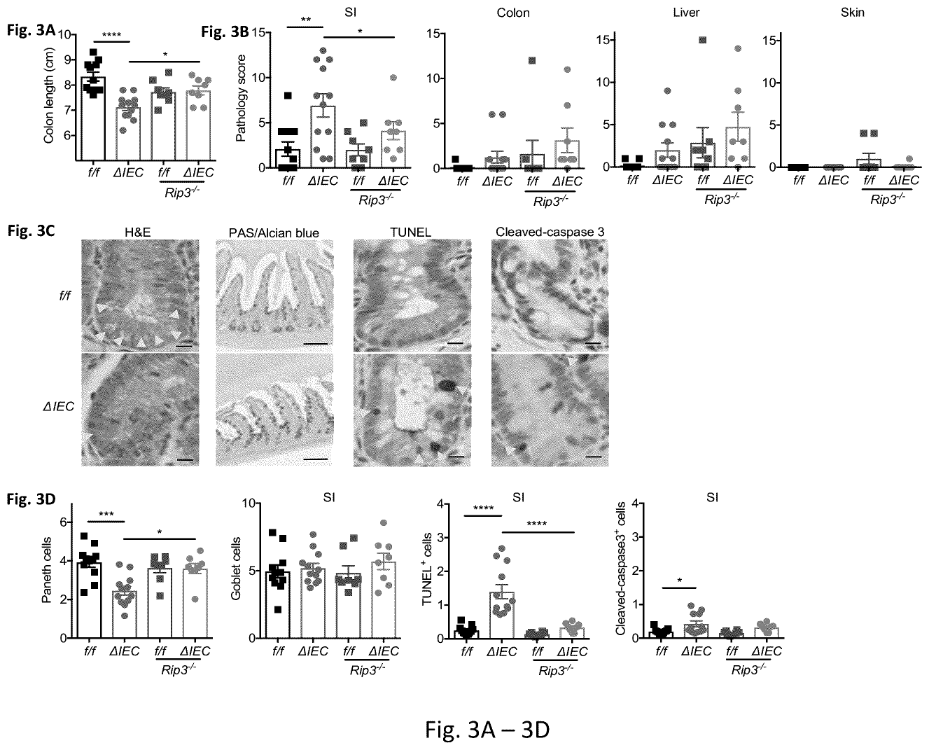

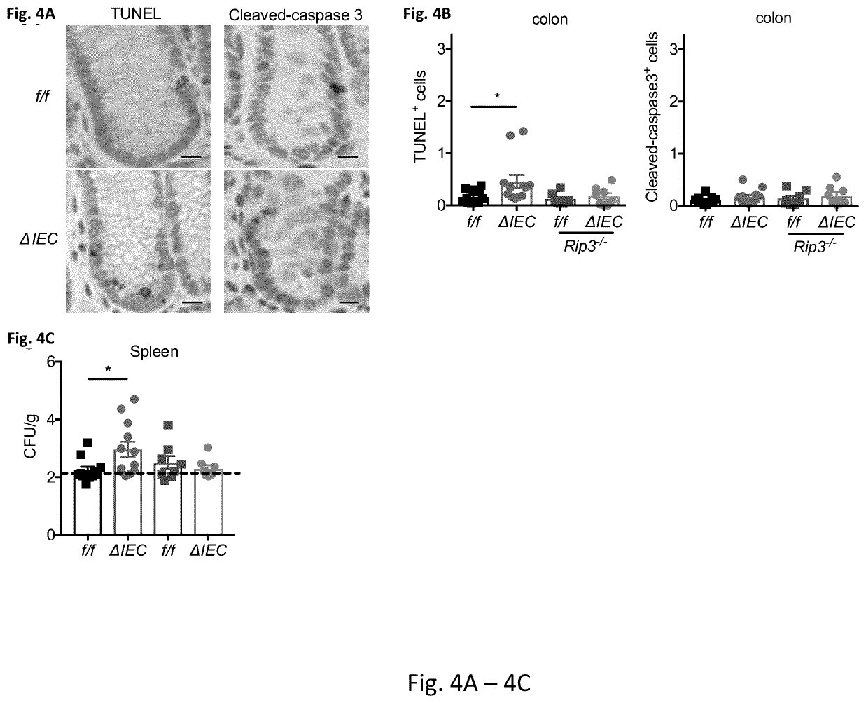

Next, the intestine was examined for signs of inflammation on day 28 after allo-HCT, the same time point at which the above immune-profiling analyses were performed. Atg16L1^{ΔIEC} mice displayed shortening of the colon (P<0.0001) compared with Atg16L1^{f/f} controls, and significantly higher histopathology scores in the small intestine (P<0.01) but not in the colon, liver, or skin (FIG. 3A and FIG. 3B). ATG16L1 has a critical role in maintaining the viability and function of Paneth cells, secretory epithelial cells in the small intestinal crypts that are identified by their characteristic antimicrobial granules (Adolph et al., 2013, Nature 503(7475):272-276; Slowicka et al., 2019, Nat Commun 10, 1834; Bel et al., 2017, Science 357, 1047-1052; Cadwell et al., Nature 456, 259-263; Cadwell et al., 2010, Cell 141, 1135-1145; Lassen et al., 2014, Proc Natl Acad Sci USA 111, 7741-7746). The importance of autophagy in organelle homeostasis may explain this role of ATG16L1. Due to their high secretory burden, Paneth cells are sensitive to ER stress (Tschurtschenthaler et al., 2017, J Exp Med. 214(2):401-422; Diamanti et al., 2017, J Exp Med. 214(2):423-437), and accumulation of mitochondria that produce reactive oxygen species (ROS) contributes to loss of viability in a RIP3-dependent manner (Matsuzawa-Ishimoto et al., 2017, J Exp Med 214, 3687-3705; Simmons et al., 2016, Cell Death Dis 7, e2196). Consistent with this literature, Paneth cells were significantly decreased in Atg16L1^{ΔIEC} (P<0.001) compared with Atg16L1^{f/f} allo-HCT recipients, whereas goblet cell numbers were similar (FIG. 3C and FIG. 3D). In certain settings, non-apoptotic cell death can be accompanied by TUNEL staining in the absence of cleaved caspase-3 (Matsuzawa-Ishimoto et al., 2017, J Exp Med 214, 3687-3705; Simmons et al., 2016, Cell Death Dis 7, e2196; Gold et al., 1994, Lab Invest 71, 219-225; Grasl-Kraupp et al., 1995, Hepatology 21, 1465-1468; Imagawa et al., 2016, Nature communications 7, 13391). The loss of Paneth cells in Atg16L1^{ΔIEC} allo-HCT recipients was associated with an increase in TUNEL⁺ cells in the crypt-base (P<0.0001), and although cleaved caspase-3 staining was not observed, the number of cells per crypt was modest (FIG. 3C and FIG. 3D). Similar results were found in the colonic epithelium of Atg16L1^{ΔIEC} mice (P<0.05), although not as striking as in the small intestine (FIG. 4A and FIG. 4B). Importantly, RIP3-deficiency reversed shortening of the colon, histopathology, Paneth cell depletion, and TUNEL-staining in Atg16L1^{ΔIEC} mice (FIG. 3A through FIG. 3D), implicating necroptosis in the intestinal inflammation that occurs in ATG16L1-deficient allo-HCT recipients. ARIP3-dependent increase in bacteria present in the spleen of Atg16L1^{ΔIEC} mice was also detected following allo-HCT (P<0.05), suggesting necroptosis of IECs causes extra-intestinal dissemination of bacteria due to intestinal barrier defects (FIG. 4C). Collectively, these data indicate that inhibition of ATG16L1 in IECs exacerbates intestinal GVHD in a RIP3-dependent manner.

Allogeneic T Cells Directly Recognize and Injure Intestinal Organoids with Autophagy Gene Mutations In Vitro

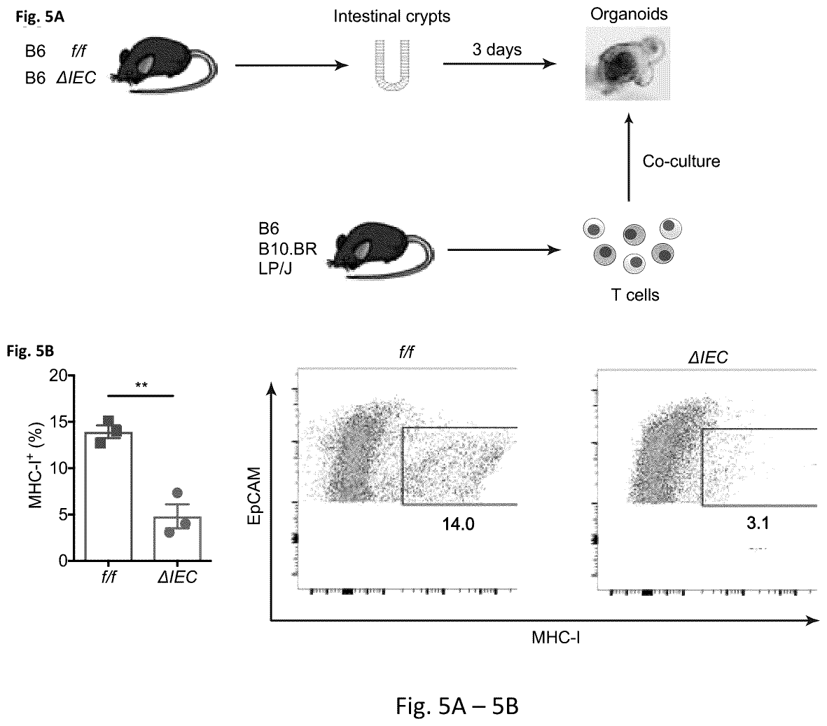

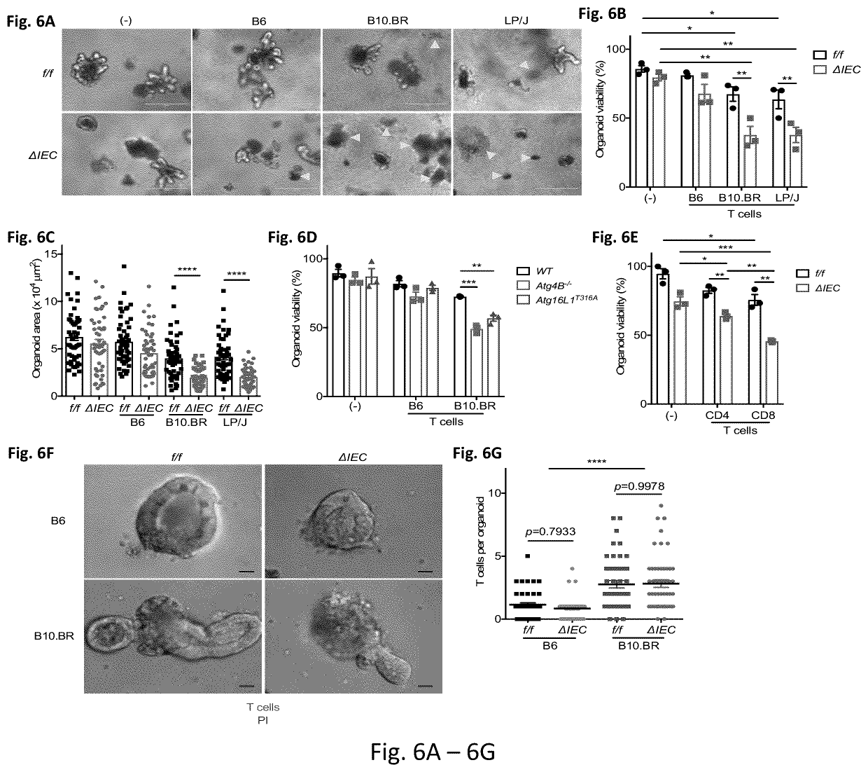

To further examine how allogeneic T cells affect IECs, and to pursue the possibility for establishing an ex vivo platform that can recreate genetic susceptibility to T cell-mediated damage, an experimental model was developed incorporating both recipient IECs and donor T cells. Primary lymphocytes from mice that are added to intestinal organoid cultures retain viability and can differentiate (Rogoz et al., 2015, J Immunol Methods 421, 89-95; Nozaki et al., 2016, J Gastroenterol 51, 206-213). However, whether such a co-culture system can be used to assess lymphocyte effector functions and cytotoxicity is unknown. An ex vivo GVHD model was established by culturing intestinal organoids together with T cells independently isolated from the spleen of allogeneic and syngeneic mice (FIG. 5A). Consistent with the findings in vivo, small intestinal organoids derived from Atg16L1^{ΔIEC} mice displayed a significant reduction in viability (P<0.01) and surface area (P<0.0001) when cultured in the presence of allogeneic T cells derived from B10.BR and LP/J donors (FIG. 6A through FIG. 6C). By comparison, T cells had a minimal effect on Atg16L1^{f/f} control organoids. The susceptibility to cell death observed in Atg16L1^{ΔIEC} organoids was dependent on alloreactivity because syngeneic T cells from B6 mice did not significantly reduce viability or size (FIG. 6A through FIG. 6C). These data indicate that alloreactivity and genetic susceptibility can be recreated ex vivo.

To confirm the findings, the susceptibility of small intestinal organoids derived from Atg4B^{-/-} mice, which are deficient in another autophagy gene, and Atg16L1^{T316A} mice, which harbor a knock-in mutation that mimics the human ATG16L1^{T300A} variant were examined. Both Atg4B^{-/-} and Atg16L1^{T316A} B6 organoids displayed impaired viability (P<0.001 (Atg4B^{-/-}) and P<0.01 (Atg16L1^{T316A})) compared with wild-type (WT) control organoids when co-cultured with B10.BR T cells (FIG. 6D). Next, to determine which population of splenic lymphocytes mediates organoid killing and to increase the purity of the cells, the organoids were co-cultured with CD4⁺ or CD8⁺ T cells sorted by flow cytometry (FACS). CD8⁺ T cells were more cytotoxic than CD4⁺ T cells, especially to the Atg16L1^{ΔIEC} organoids (P<0.01) (FIG. 6E). T cells from B10.BR donors, but not syngeneic B6 donors, were physically associated with organoids when examined by light microscopy (P<0.0001) (FIG. 6F and FIG. 6G). Autophagy can suppress MHC class-I (MHC-I) levels in dendritic cells, thus raising the possibility that Atg16L1-deficiency confers susceptibility due to an increase in MHC-I. However, surface MHC-I was lower rather than higher in Atg16L1^{ΔIEC} organoids (P<0.01) compared with Atg16L1^{f/f} controls (FIG. 5B), indicating a cell type-specific role of autophagy in inhibiting surface MHC-I levels. Additionally, the number of T cells associated with Atg16L1^{f/f} and Atg16L1^{ΔIEC} organoids were similar, suggesting that genotype is unlikely to influence the initial recognition of IECs by allogeneic T cells (FIG. 6F and FIG. 6G). Collectively, these data suggest that Atg16L1-deficiency causes organoids to become susceptible to the cytotoxic activity of allogeneic T cells, especially CD8⁺ T cells.

Allogeneic T cells Induce TNFα-Mediated Necroptosis in ATG16L1-Deficient Intestinal Organoids

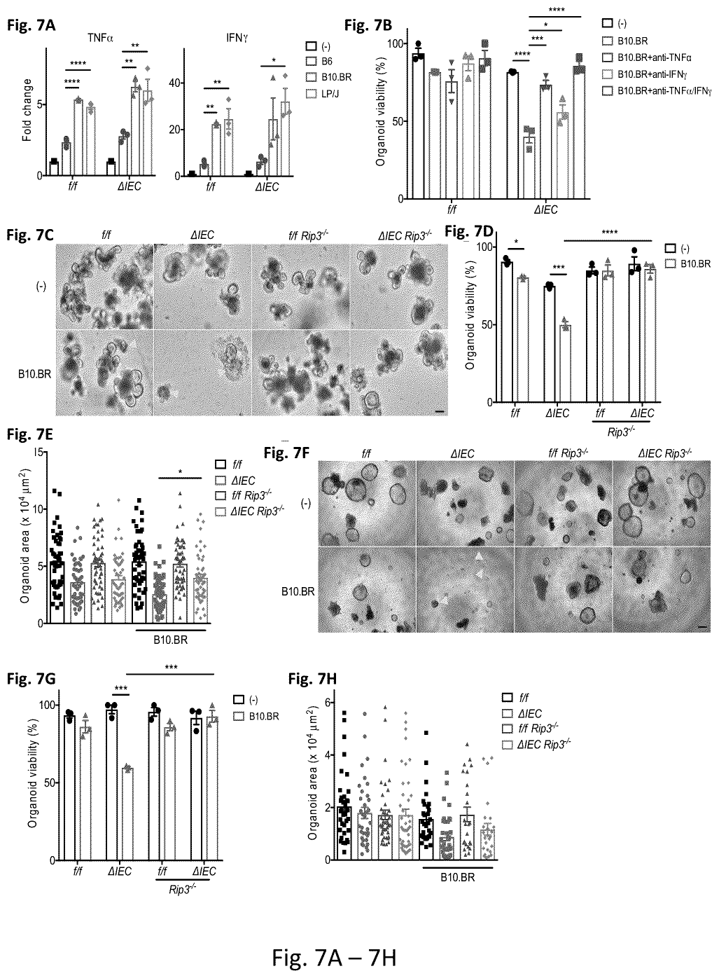

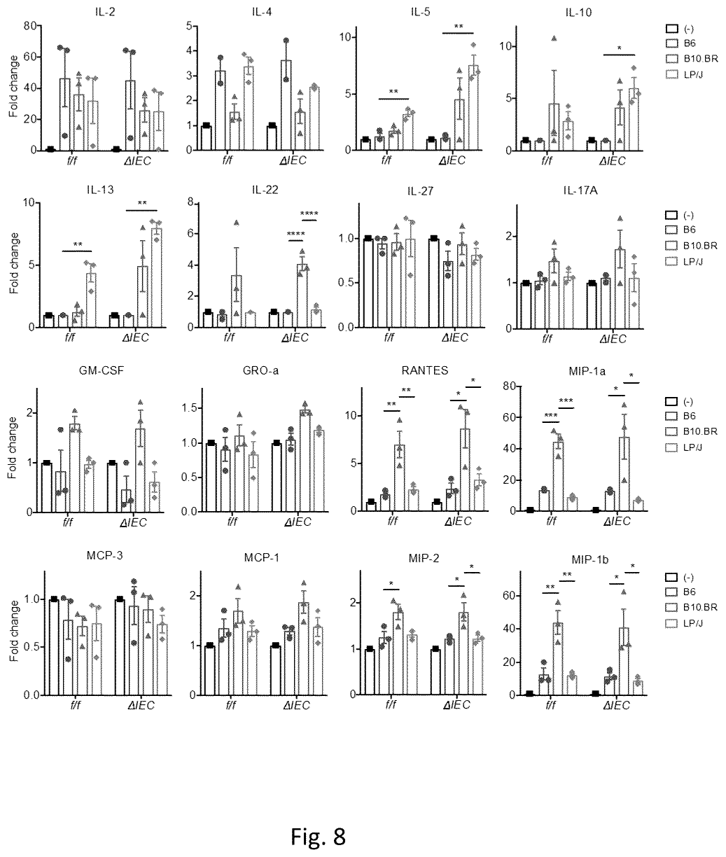

Next, the mechanism by which allogeneic T cells injure the Atg16L1^{ΔIEC} organoids was investigated. Because various cytokines were reported to be cytotoxic to Atg gene mutant organoids (Matsuzawa-Ishimoto et al., 2017, J Exp Med 214, 3687-3705; Burger et al., 2018, Cell Host Microbe 23, 177-190 e174; Aden et al., 2018, J Exp Med 215, 2868-2886), a panel of soluble factors was quantified in the culture supernatants from the organoid-T cell co-culture. Both Atg16L1^{f/f} and Atg16L1^{ΔIEC} organoid samples containing allogeneic B10.BR or LP/J T cells had higher level of TNFα and IFNγ compared with supernatant from organoids cultured with syngeneic B6 T cells or no T cells (FIG. 7A). Organoids, especially Paneth cells, have been shown to be sensitive to these two cytokines (Matsuzawa-Ishimoto et al., 2017, J Exp Med 214, 3687-3705; Farin et al., 2014, J Exp Med 211, 1393-1405; Eriguchi et al., 2018, JCI Insight 3, 121886). Supernatant from organoids co-cultured with B10.BR T cells, but not the other conditions, contained higher levels of IL-22 (FIG. 8), which has been shown to exacerbate necroptosis in Atg16L1^{ΔIEC} organoids (19). The effect of adding blocking antibodies against TNFα and IFNγ to the culture was tested because these two cytokines were produced in both the B10.BR and LP/J-containing conditions. Blocking TNFα significantly increased survival of Atg16L1^{ΔIEC} organoids (P<0.001), blocking IFNγ modestly improved survival (P<0.05), and blocking TNFα and IFNγ together completely rescued viability (P<0.0001) (FIG. 7B). Because RIP3-deficiency protected Atg16L1^{ΔIEC} mice from GVHD, both small intestinal and colonic organoids were generated from Atg16L1^{f/f} Rip3^{-/-} and Atg16L1^{ΔIEC} Rip3^{-/-} mice, and co-cultured them with B10.BR T cells. RIP3-deficiency protected small intestinal and colonic Atg16L1^{ΔIEC} organoids from B10.BR T cell-mediated injury (P<0.0001 (small intestine) and P<0.001 (colon)) (FIG. 7C through FIG. 7H). These data are highly consistent with the in vivo results and together support a model in which allogeneic T cells producing inflammatory cytokines induce necroptosis in ATG16L1-deficient IECs.

Loss of Viability in ATG16L1-Deficient Intestinal Organoids is Associated with A Type-I Interferon Signature

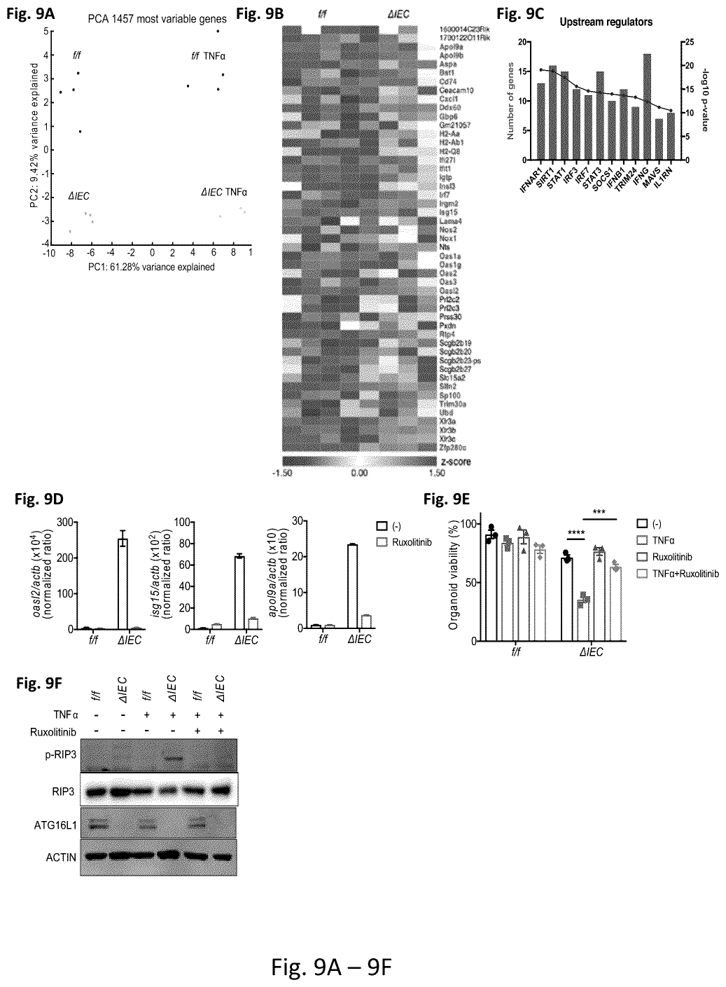

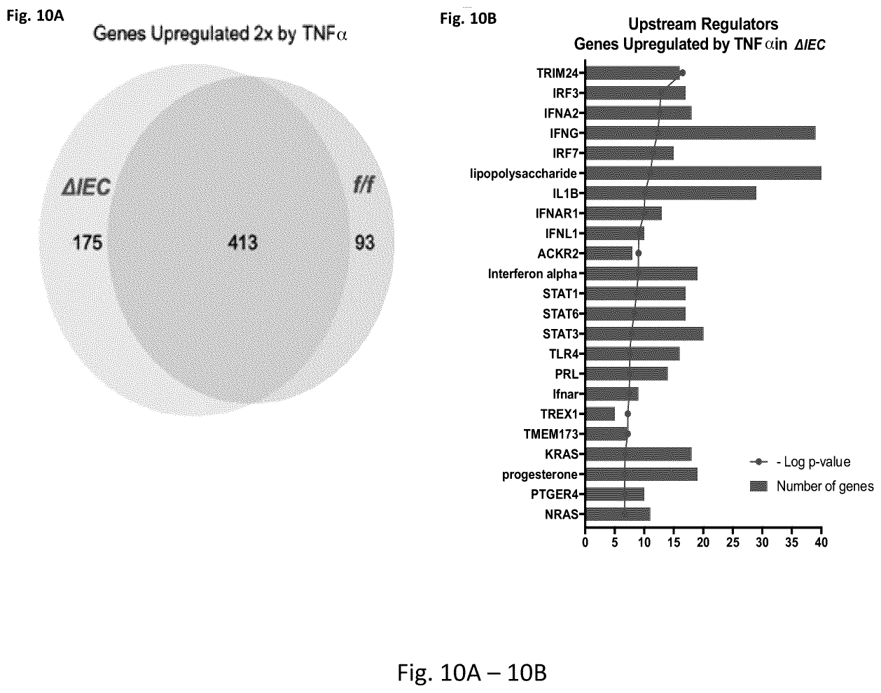

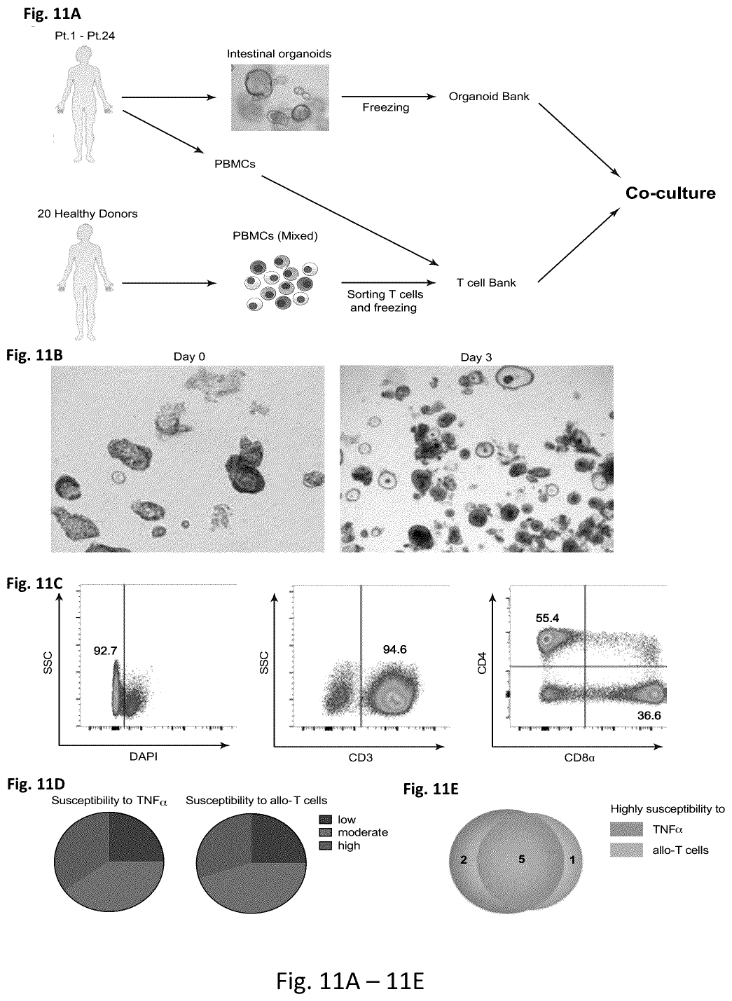

To examine the mechanism by which ATG16L1-deficiency renders IECs susceptible to necroptosis, RNA Sequencing analysis was performed using Atg16L1^{f/f} and Atg16L1^{ΔIEC} small intestinal organoids, with or without TNFα treatment. Principal component analysis (PCA) shows the samples cluster according to their condition, with cytokine stimulation and genotype statuses separating along PC1 and PC2, respectively (FIG. 9A). In the absence of TNFα, 49 genes were upregulated fold in Atg16L1^{ΔIEC} over Atg16L1^{f/f} organoids, many of which were known interferon-stimulated genes (ISGs) (40) (FIG. 9B). Indeed, pathway analysis of this gene set confirmed a type-I interferon (IFN-I) signature in Atg16L1^{ΔIEC} organoids (FIG. 9C). ISGs remained upregulated in Atg16L1^{ΔIEC} organoids treated with TNFα (FIG. 10A and FIG. 10B). Increased expression of genes associated with cytokine receptor signaling in TNFα-treated organoids was also found, but most of these were not significantly impacted by Atg16L1 deficiency (FIG. 10A and FIG. 10B). These data are consistent with recent finding that intestinal tissue from mice with reduced Atg16L1 expression have an increase in ISG expression and pSTAT1⁺ IECs (Martin et al., 2018, Nat Microbiol 3, 1131-1141).

IFN-I signals through IFNAR1 to activate JAK1 and STAT1/2 leading to expression of ISGs with broad functions, frequently associated with antiviral immunity (Rauch et al., 2013, JAKSTAT 2, e23820). The IFN-I response also has been shown to intersect necroptosis and TNFα signaling pathways (Robinson et al., 2012, Nat Immunol 13, 954-962; Thapa et al., 2013, Proc Natl Acad Sci USA 110, E3109-3118; Lin et al., 2016, Nature 540, 124-128; Newton et al., 2016, Nature 540, 129-133; Kuriakose et al., 2016, Sci Immunol 1, aag2045; Lim et al., 2019, Elife 8, e44452; Hos et al., 2017, J Cell Biol 216, 4107-4121; Legarda et al., 2016, Cell Rep 15, 2449-2461; Sarhan et al., 2019, Cell Death Differ 26, 332-347; Li et al., 2018, Cell Death Differ 25, 1304-1318). Necroptosis dependent on RIP3 and JAK/STAT signaling downstream of IFN-I promote immunity during viral infection, providing an explanation as to why an antiviral cytokine is associated with an inflammatory form of programmed cell death (Kuriakose et al., 2016, Sci Immunol 1, aag2045; Upton et al., 2012, Cell Host Microbe 11, 290-297; Thapa et al., 2016, Cell Host Microbe 20, 674-681). Although the role of IFN-I in GVHD is complex because it can act on both the T cells and the target tissue at multiple steps of the allo-HCT procedure (Robb et al., 2011, Blood 118, 3399-3409; Fischer et al., 2017, Sci Transl Med 9, eaag2513), recent studies reported the efficacy of JAK-STAT inhibitors including Ruxolitinib in ameliorating GVHD in animal models and patients (Schroeder et al., 2018, Biol Blood Marrow Transplant 24, 1125-1134; Spoerl et al., 2014, Blood 123, 3832-3842). Therefore, whether Ruxolitinib can improve the viability of Atg16L1^{ΔIEC} organoids was tested. First it was confirmed that Atg16L1^{ΔIEC} organoids display an IFN-I signature by showing that three representative ISGs (Oas12, Isg15, and Apo19a) are expressed higher compared with Atg16L1^{f/f} organoids, and it was found that the expression of these genes can be inhibited by Ruxolitinib treatment (FIG. 9D). It was found that treatment with Ruxolitinib also protected Atg16L1^{ΔIEC} organoids from TNFα-induced death (P<0.001) (FIG. 9E), and the increased level of phosphorylated (p)-RIP3 in TNFα-treated Atg16L1^{ΔIEC} organoids that coincides with necroptosis was also restricted with Ruxolitinib (FIG. 9F). These data indicate that JAK-STAT signaling in ATG16L1-deficient organoids contribute to the susceptibility to TNFα-mediated necroptosis, and suggest that Ruxolitinib may act in part by protecting intestinal tissue in addition to direct effects on T cells (Spoerl et al., 2014, Blood 123, 3832-3842).

Development of An Ex Vivo Intestinal GVHD Model Using Human Intestinal Organoids and Peripheral T cells

Next, whether human organoids display loss of viability when co-cultured with T cells from human donors was examined. Intestinal organoids were generated from endoscopic biopsy specimens (FIG. 11A). Most biopsies were collected from Crohn's disease patients because of their high probability of harboring the ATG16L1′ 0A risk allele and the availability of small intestinal biopsies. Viable organoids can be generated from frozen tissue, allowing parallel experiments with banked immune cells (Konnikova et al., 2018, Mucosal Immunol 11, 1684-1693), or in this case, co-culture. T cells were sorted from peripheral blood mononuclear cells (PBMCs) obtained from either the same individuals as above or from an independent cohort of 20 healthy donors. To accurately compare viability in the presence of alloreactive T cells, it was necessary to establish a system in which all organoids were cultured in the presence of the same set of donor T cells. Therefore, PBMCs from the healthy donors were mixed prior to sorting T cells (FIG. 11A). Prior to performing the following co-culture experiments, it was confirmed that thawed organoids proliferated well in the absence of stimuli (FIG. 11B), as well as the viability and purity of isolated T cells (FIG. 11C). As an additional condition, the susceptibility of organoids to recombinant human TNFα was simultaneously evaluated.

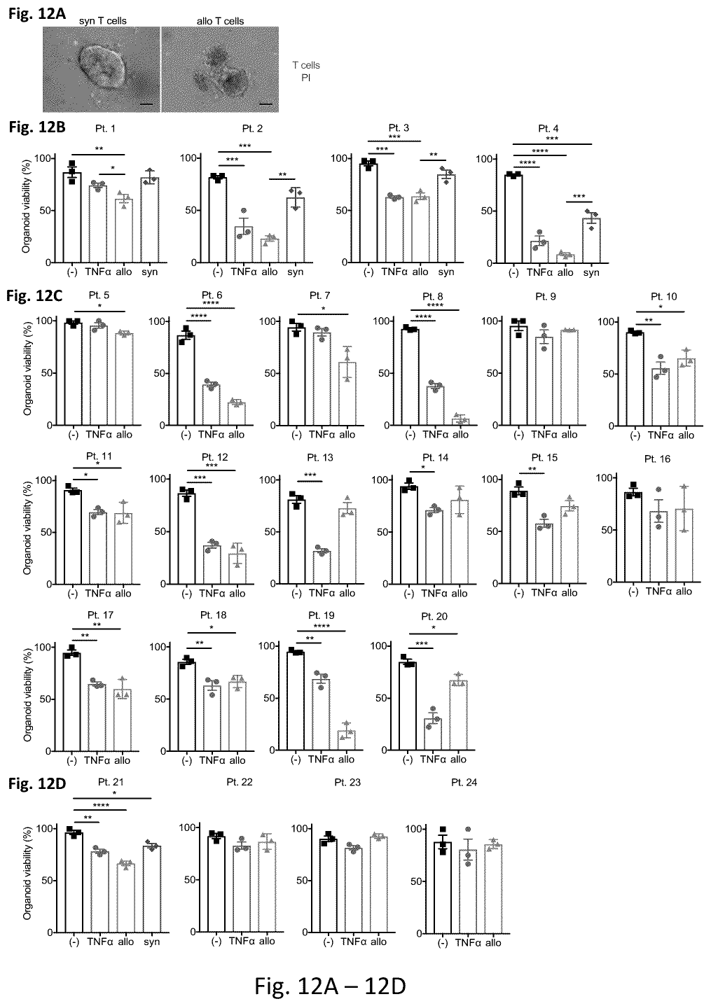

Consistent with findings in the mouse model, co-cultured allogeneic T cells were generally more toxic to the human-derived organoids than syngeneic ones (FIG. 12A and FIG. 12B). Further, there was substantial variability in susceptibility to TNFα or allogeneic T cells. Among the 20 small intestinal organoids tested, 15 displayed a statistically significant reduction in viability (P<0.05) (75%) in the presence of allogeneic T cells and 6 displayed a high degree of susceptibility (30%) as defined as >50% loss in viability (FIG. 12B and FIG. 12C, and FIG. 12D). Similarly, 15 displayed a statistically significant reduction in viability (P<0.05) (75%) in the presence of recombinant human TNFα and 7 displayed a high degree of susceptibility (46.7%) (FIG. 12B and FIG. 12C, FIG. 11D). Although organoids that were susceptible to allogeneic T cells were generally susceptible to TNFα and vice versa, there were several examples in which individual organoids displayed noticeable differences in viability between these two treatments (patients #13, 19, 20) (FIG. 11E). Together, these findings establish a model to test IEC resilience to immune-mediated injury, and show that organoids derived from the small intestine display variability in susceptibility to killing by allogeneic T cells and TNFα.

In Atg16L1^{ΔIEC} allo-HCT recipient mice, the small intestine displayed increased numbers of IECs that were TUNEL⁺ compared with the colon. Human organoids derived from the colon were relatively resistant to killing by T cells or TNFα (FIG. 12D). Thus, the selective sensitivity of small intestinal epithelial cells appears to be a conserved feature in mice and humans.

Intestinal Organoids Derived from ATG16L1^{T300A} Homozygous Individuals Display Heightened Susceptibility to TNFα and Allogeneic T cells

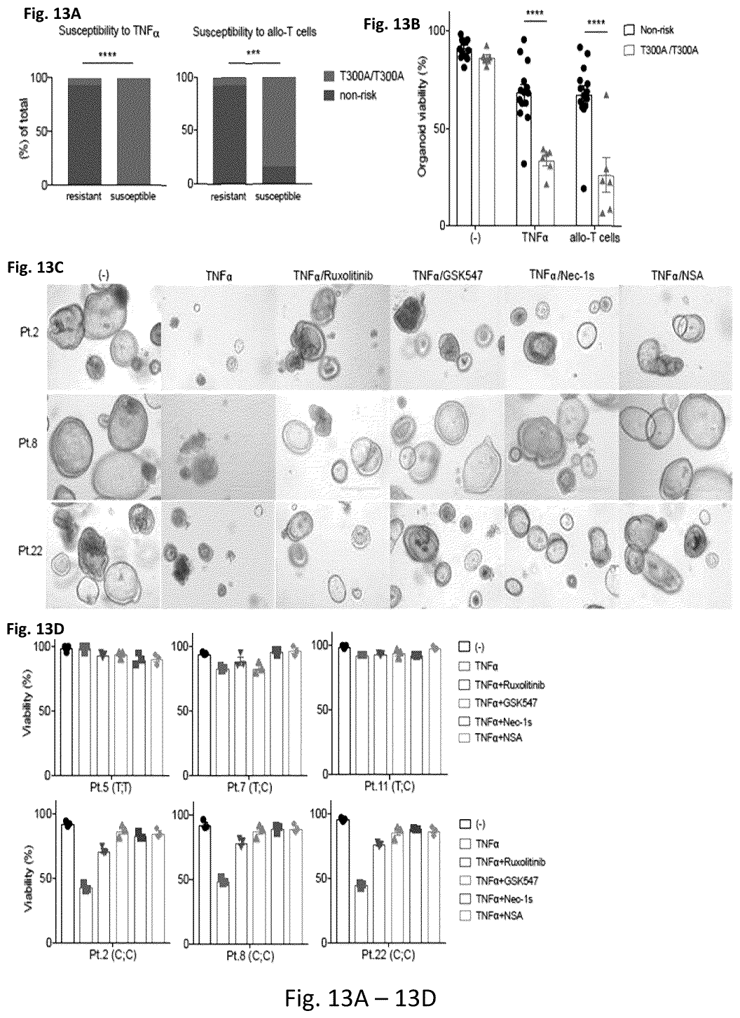

Given that mouse organoids derived from Atg16L1^{ΔIEC} and Atg16L1^{T316A} mice were susceptible to allogeneic T cell-mediated injury (FIG. 12A through FIG. 12D), it was hypothesized that the presence of the ATG16L1^{T300A} risk allele contributes to the variability in the outcome of the experiments using human organoids. The samples from the previous experiment were retrospectively genotyped for the presence of common single nucleotide polymorphisms (SNPs). Remarkably, almost all of the small intestinal organoids that displayed >50% death in the presence of TNFα or allogeneic T cells were derived from individuals harboring 2 copies of the ATG16L1^{T300A} risk variant (r52241880), whereas the majority of resistant organoids were from individuals with 0 or 1 copy of the allele (P<0.0001 (TNFα) and P<0.001 (allogeneic T cells)) (FIG. 13A). It was observed that several other IBD risk variants were present in the cohort, such as NOD2^{R702W} (r52066844), LRRK2^{N2081D} (r533995883), and IRGM (r513361189). However, organoids harboring these other risk variants were resistant to both stimuli (FIG. 12B and FIG. 12C). Analysis of the dataset by comparing the degree of viability between individuals with 2 copies versus 0 or 1 copy of ATG16L1^{T300A} supported the conclusion that organoids from individuals who are homozygous for this allele display significantly reduced survival in the presence of either TNFα or allogeneic T cells (P<0.0001) (FIG. 13B).

Finally, whether drugs that target the underlying mechanism of susceptibility based on the mouse model would reverse the selective defect in viability displayed by ATG16L1^{T300A} homozygous human organoids was examined. Specifically, the efficacy of two RIP1 inhibitors (Necrostatin-1s and GSK547), an MLKL inhibitor (Necrosulfonamide; NSA), and Ruxolitinib were tested. Susceptibility to TNFα was used as the assay rather than allogeneic T cells to avoid potential confounding effect of the drugs on T cells; Ruxolitinib in particular is known to suppress T cell function (Keohane et al., 2015, Br J Haematol 171:60-73). At concentrations that are non-toxic to organoids from non-risk patients, all 4 of these inhibitors significantly protected ATG16L1^{T300A} homozygous organoids from TNFα-induced death (P<0.0001) (FIG. 13C and FIG. 13D). These data indicate that ATG16L1 protects not only mouse but also human IECs from TNFα-mediated necroptosis, and that necroptosis and JAK-STAT inhibitors could be promising therapeutic options for intestinal GVHD in patients with ATG16L1^{T300A} risk alleles.

### Example 2: Anti-TNFα Responsiveness in Subjects with Ulcerative Colitis

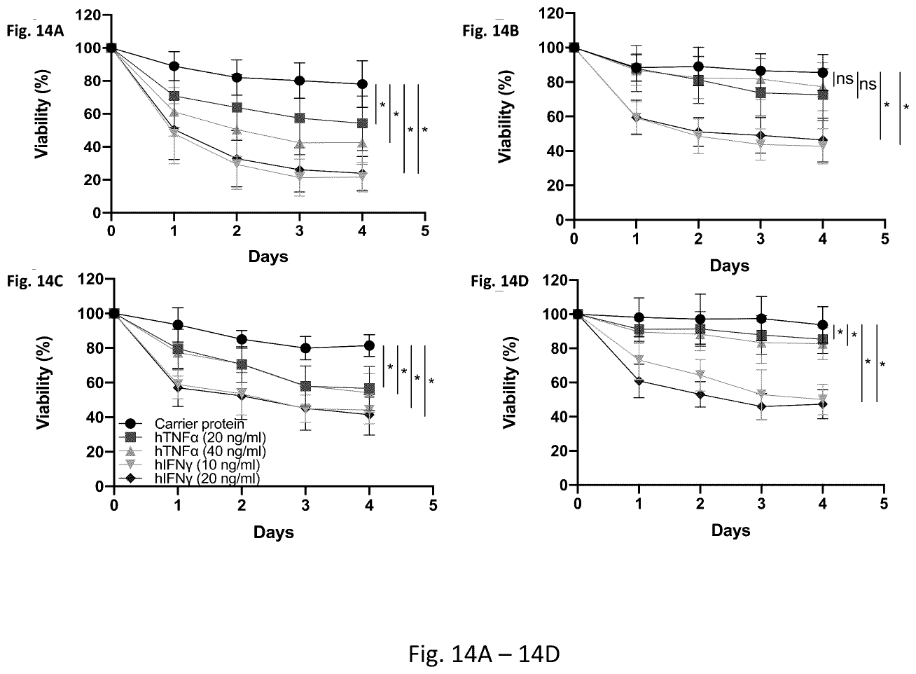

Experiments were conducted using organoids derived from subjects with ulcerative colitis (UC) to examine their susceptibility to TNFα-mediated injury. Viability over time of organoids from 18 UC patients (9 naive, 4 responsive, and 5 refractory to anti-TNFα) was measured by microscopy following treatment with 20 or 40 ng/ml recombinant human TNFα. Organoids were also treated with 10 or 20 ng/ml human interferon gamma (IFNγ) as a control cytokine expected to be toxic. FIG. 14A demonstrates that organoids derived from anti-TNF-responsive patients were susceptible TNFα, as measured by viability, while FIG. 14B demonstrates that organoids derived from anti-TNF-refractory patients were resistant to TNFα. FIG. 14C and FIG. 14D demonstrates that organoids derived from anti-TNF-naive patients could be divided into either susceptible (FIG. 14C) or resistant groups (FIG. 14D). These results indicate that organoids from UC patients can be segregated based on their sensitivity to cytokines, which is reflective of the patient's clinical responsiveness to treatments. Further, these results demonstrate the predictive value of intestinal organoid cultures and clinical utility.

### Example 3: IL-17 Treatment Identified Responsive and Unresponsive Organoids

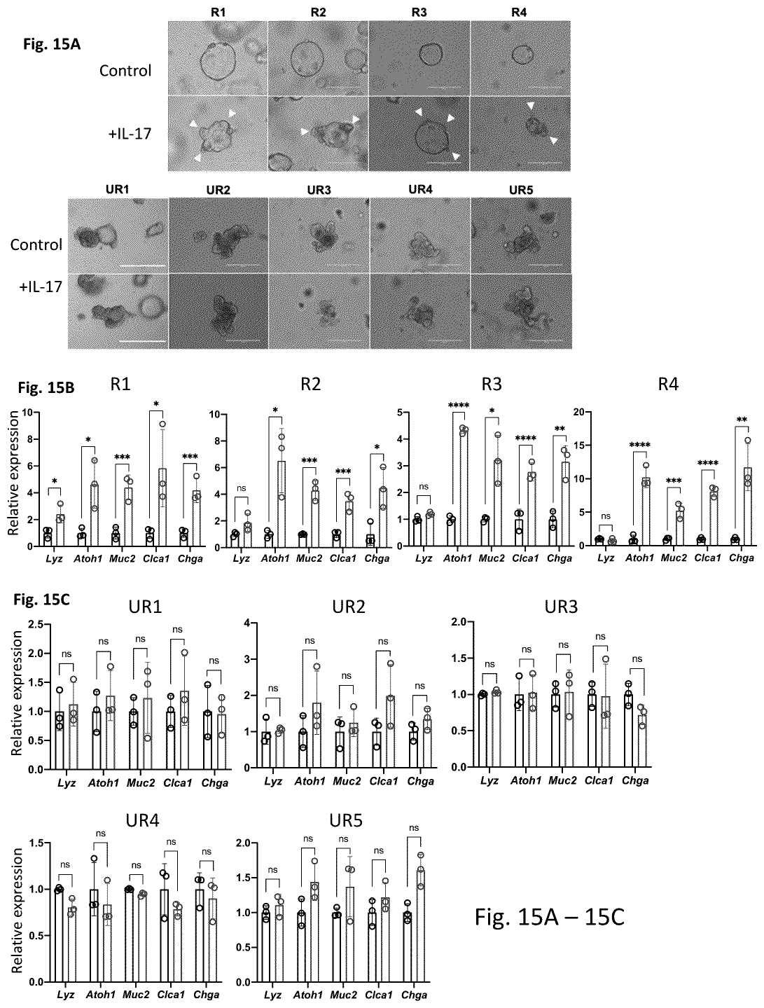

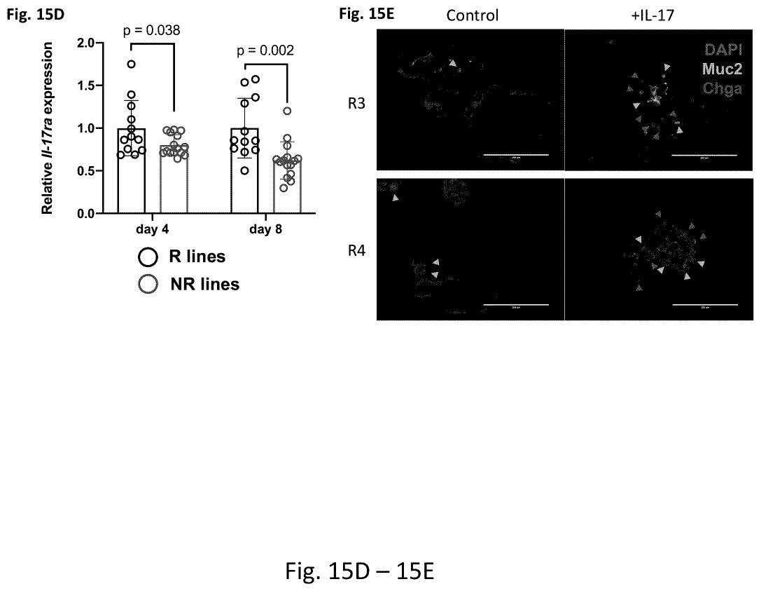

Experiments were conducted using organoids derived from individuals to examine the responsiveness to IL-17 treatment. Intestinal organoids from small intestinal biopsies procured from nine individuals were differentiated in the presence of 10 ng/ml of the cytokine IL-17A. Four of the nine organoids (R1-R4) responded to IL-17A treatment by converting from cystic morphology to displaying buds, a sign of enhanced differentiation of secretory epithelial cells (FIG. 15A). In contrast, five out of the nine were unresponsive (UR1-UR5) and displayed similar morphology when comparing IL-17A treated and control carrier protein only (FIG. 15B). Unresponsive organoids were characterized by budding in the absence of IL-17A. FIG. 15B depicts quantitative RT-PCR (qPCR) analysis which indicates that organoids identified as responsive display enhanced expression of the indicated genes associated with secretory epithelial cells: LYZ (Paneth cells), ATOH1 (secretory lineage commitment), MUC2 and CLCA/(goblet cells), and CHGA (enteroendocrine cells). qPCR analysis of unresponsive lines indicates that these lineage markers are not altered in these organoids (FIG. 15C). Additionally, it was found that responsive lines are characterized by higher expression of the receptor for IL-17 (IL-17RA) (FIG. 15D).

Gene expression results were validated by staining sections of representative responsive organoids (R3 and R4) for MUC2 and CHGA at the protein level with antibodies and visualizing by fluorescent microscopy on day 8 post IL-17A treatment (FIG. 15E). These results indicate that organoids display distinct morphologies, which can be further affected by the immune effector molecule IL-17A. Organoids that are sensitive to IL-17A respond through enhanced differentiation of secretory cell lineage. Further, these results support the idea that the invention can be used to examine immune interactions and heterogeneity among patients.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating or preventing a disease or disorder associated with immune response-mediated tissue injury in a subject in need thereof, the method comprising:
identifying the subject as having an inactivating mutation in the Autophagy Related 16 Like 1 gene (ATG16L1), and
administering to the subject at least one inhibitor selected from the group consisting of an inhibitor of necroptosis and an inhibitor of interferon signaling.

2. The method of claim 1, wherein the inactivating mutation in ATG16L1 is a T300A mutation.

3. The method of claim 1, wherein the subject has been diagnosed with at least one disease or disorder selected from the group consisting of intestinal graft-versus-host disease (GVHD), inflammatory bowel disease (IBD), Crohn's disease (CD), ulcerative colitis (UC), pouchitis, irritable bowel syndrome (IBS), infectious and non-infectious gastroenteritis, autoimmunity associated with cancer immunotherapy, gastrointestinal cancer, and radiation enteritis.

4. The method of claim 1, wherein the inhibitor is selected from the group consisting of a chemical compound, a protein, a peptide, a peptidomimetic, an antibody, a ribozyme, a small molecule chemical compound, a nucleic acid, a vector, and an antisense nucleic acid molecule.

5. The method of claim 4, wherein the inhibitor is an inhibitor of at least one selected from the group consisting of RIPK1, RIPK3, MLKL and JAK/STAT.

6. The method of claim 5, wherein the inhibitor is an RIPK1 inhibitor selected from the group consisting of a Necrostatin, Vorinostat, 1-Benzyl-1H-pyrazole derivatives, aminoisoquinolines, PN10, Cpd27, GSK′840, GSK′843, GSK′872, Curcumin, tozasertib, ponatinib, pazopanib, GSK2982772, DNL747 and small molecule necroptosis inhibitors and analogs and derivatives thereof.

7. The method of claim 5, wherein the inhibitor is an RIPK3 inhibitor selected from the group consisting of GSK′840, GSK′843, GSK′872, Ganoderma lucidium Mycelia, Kongensin A, Celastrol, ponatinib, HS-1371, dabrafenib and analogs and derivatives thereof.

8. The method of claim 5, wherein the inhibitor is an MLKL inhibitor selected from the group consisting of ponatinib, pazopanib, necrosulphonamide, Compound 1, Celastrol, TC13172, and analogs and derivatives thereof.

9. The method of claim 5, wherein the inhibitor is a JAK/STAT inhibitor selected from the group consisting of tofacitinib, ruxolitinib, peficitinib, filgotinib, solcitinib, upadacitinib, baricitinib, itacitinib, SHR0302, PF04965842, decernotinib and analogs and derivatives thereof.

10. The method of claim 1, wherein the necroptosis inhibitor is selected from the group consisting of furo[2,3-d]pyrimidine, pyrrolo[2,3-b]pyridines, IM-54, a NecroX analog, GSK2982772, Terminalia Chebula, Naringenin, a small molecule necroptosis inhibitor, a tricyclic necrostatin compound, a heterocyclic inhibitor of necroptosis, a spiroquinoxaline derivative, tofacitinib, ruxolitinib, peficitinib, filgotinib, solcitinib, and upadacitinib, and analogs and derivatives thereof.

11. A method for preparing an intestinal organoid culture, wherein the method comprises:
culturing small intestinal and colonic crypt cells in contact with an extracellular matrix to obtain an intestinal organoid;
removing said extracellular matrix from said intestinal organoids;
and re-suspending said intestinal organoids in a medium.

12. The method of claim 11, wherein the medium comprises at least one additional agent selected from the group consisting of an immune cell and an inflammatory cytokine.

13. The method of claim 11, wherein the small intestinal and colonic crypt cells are cultured in a medium comprising mEGF, mNoggin and mR-Spondin 1 (ENR medium).

14. The method of claim 12, wherein the immune cells are T cells.

15. The method of claim 12, wherein the small intestinal and colonic crypt cells and immune cells are obtained from the same subject.

16. An intestinal organoid culture obtained by the method of claim 11.

17. A method for testing a therapeutic agent, wherein the method comprises:
contacting an intestinal organoid culture of claim 16 with one or more candidate agents,
detecting the presence or absence of one or more change in the intestinal organoid culture that is indicative of therapeutic efficacy, and
identifying a candidate agent as a therapeutic agent if the presence or absence of one or more of said changes in the intestinal organoid culture is detected.

18. The method of claim 17, wherein the said change in the intestinal organoid co-culture is selected from the group consisting of an increase in cell viability, organoid size, morphology, quantification of epithelial subsets, cell proliferation, transcriptome, protein levels or post-translational modifications of proteins, metabolism, production of soluble factors and any combination thereof of the intestinal organoid cells as compared to a comparator control.

19. The method of claim 17, wherein the therapeutic agent is suitable for the treatment of a disease or disorder associated with immune response-mediated tissue injury.

20. The method of claim 19, wherein the disease or disorder associated with immune response-mediated tissue injury is selected from the group consisting of GVHD, IBD, CD, UC, pouchitis, IBS, infectious and non-infectious gastroenteritis, autoimmunity associated with cancer immunotherapy, gastrointestinal cancer, and radiation enteritis.


SEQUENCE LISTING (8 sequences)

SEQ ID NO: 1 - DNA, 20 bp, synthetic construct
  misc_feature 1..20: note = Chemically synthesized
  source 1..20: mol_type = other DNA
ggatgcctgggagagaatcg

SEQ ID NO: 2 - DNA, 20 bp, synthetic construct
  misc_feature 1..20: note = Chemically synthesized
  source 1..20: mol_type = other DNA
tcgcctgctcttcgaaactg

SEQ ID NO: 3 - DNA, 21 bp, synthetic construct
  misc_feature 1..21: note = Chemically synthesized
  source 1..21: mol_type = other DNA
ggtgtccgtgactaactccat

SEQ ID NO: 4 - DNA, 21 bp, synthetic construct
  misc_feature 1..21: note = Chemically synthesized
  source 1..21: mol_type = other DNA
tggaaagggtaagaccgtcct

SEQ ID NO: 5 - DNA, 21 bp, synthetic construct
  misc_feature 1..21: note = Chemically synthesized
  source 1..21: mol_type = other DNA
gtggataggattgccagcaag

SEQ ID NO: 6 - DNA, 21 bp, synthetic construct
  misc_feature 1..21: note = Chemically synthesized
  source 1..21: mol_type = other DNA
agaggggttcctttcagactg

SEQ ID NO: 7 - DNA, 24 bp, synthetic construct
  misc_feature 1..24: note = Chemically synthesized
  source 1..24: mol_type = other DNA
cggttccgatgccctgaggctctt

SEQ ID NO: 8 - DNA, 24 bp, synthetic construct
  misc_feature 1..24: note = Chemically synthesized
  source 1..24: mol_type = other DNA
cgtcacacttcatgatggaattga